(19) United States
(12) Patent Application Publication
(10) Pub. No.: US 2012/0115244 A1
(43) Pub. Date: May 10, 2012

(54) MATERIALS AND METHODS FOR IMMUNOASSAY OF PTERINS

(75) Inventors: Maciej Adamczyk, Gurnee, IL (US); R. Jeffrey Brashear, Mundelein, IL (US); Stephen C. Hsu, Buffalo Grove, IL (US); Phillip G. Mattingly, Third Lake, IL (US)

(73) Assignee: ABBOTT LABORATORIES, Abbott Park, IL (US)

(21) Appl. No.: 12/942,781

(22) Filed: Nov. 9, 2010

(51) Int. Cl. G01N 33/53; C07K 14/765; C07K 2/00; C07K 14/435; C07K 16/00; C07D 475/04

(52) U.S. Cl. 436/501; 530/391.3; 544/258; 530/405; 530/386; 530/363

(57) ABSTRACT

Methods of assaying for (i) a pterin by immunoassay employing a pterin as capture agent, (ii) neopterin by chemiluminescent microparticle immunoassay (CMIA) employing an anti-neopterin antibody (Ab) as capture agent, (iii) neopterin by an immunoassay (IA) employing an acridinium (Acr)-labeled anti-neopterin Ab as conjugate, and (iv) neopterin by an IA employing Acr-labeled neopterin as tracer; an Acr-labeled anti-neopterin Ab; a conjugate/complex comprising anti-neopterin Ab and a carrier scaffold; a conjugated pterin; a conjugate comprising an Acr-labeled pterin and a carrier scaffold; an immunogen comprising neopterin and a carrier protein; a conjugate comprising such an immunogen and an Acr compound; an immunogen comprising a carrier protein and a neopterin hapten; a conjugate comprising such an immunogen and an Acr compound; a kit for assaying a pterin comprising a pterin as a capture agent and instructions for IA; and a kit for assaying neopterin comprising an anti-neopterin Ab as a capture agent and instructions for CMIA, neopterin comprising an Acr-labeled anti-neopterin Ab as a conjugate and instructions for IA, or Acr-labeled neopterin as a tracer and instructions for IA.

### TECHNICAL FIELD

This disclosure relates to pterins, specifically neopterin, antibodies, conjugates/complexes, immunogens, immunoassays, and kits.

### BACKGROUND

Monocyte/macrophage activation and inflammation are accompanied by an increase in neopterin, which is a derivative of pteridine and a byproduct of the guanosine triphosphate-biopterin pathway. Neopterin has the chemical structure:

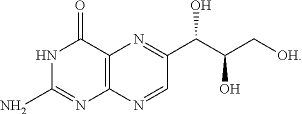

Neopterin is also known as D-erthryo-neopterin and 2-amino-6-[(1S,2R)-1,2,3-trihydroxypropyl]-4(3H)pteridinone. It has the formula C₉H₁₁N₅O₄, a molecular weight of 253.21, and the CAS Reg. No. [2009-64-5]. Neopterin is biochemically inert, has a long biological half-life, is exclusively synthesized in and released from activated macrophages, is eliminated by the kidneys (Berdowska et al., J. Clin. Pharm. Ther. 26(5): 319-329 (October 2001); see, also, review by Hamerlinck, Exp. Dermatol. 8: 167-176 (1999)), and is easy to measure in serum, plasma, urine, cerebrospinal fluid, etc. Serum levels above 10 nmol/L are generally regarded as elevated (Berdowska et al. (2001), supra; see, also, U.S. Pat. App. Pub. No. 2006/0063162 regarding neopterin as a marker of inflammation and U.S. Pat. App. Pub. No. 2006/0166270 regarding neopterin as a marker of demyelinization). In contrast, interferon-γ (IFNγ) is a homodimeric 50 kDa Th-1 cytokine, which rapidly binds to target receptors, has a short biological half-life, is synthesized in and released from CD4+/CD8+ T-cells and NK cells, is an indicator of systemic immune system activation, and, consequently, is not a good target for routine laboratory diagnosis.

Increased neopterin concentrations in bodily fluids, such as serum or urine, are connected with diseases involving a cellular immune reaction (Fuchs et al., Immunol. Today 9: 150-155 (1988); Wachter et al., Adv. Clin. Chem. 27: 81-141 (1989); Fuchs et al., Crit. Rev. Clin. Lab. Sci. 29: 304-341 (1992); Fuchs et al., Int'l Arch Allergy Immunol. 101: 1-6 (1993); Wachter et al., Neopterin: Biochemistry—Methods—Clinical Application, Walter deGruyter, Berlin, N.Y., 1992; and Fuchs et al., In: Labor and Diagnose, Thomas, L., ed., Die Medizinische Verlagsgesellschaft, Marburg/Lahn, 1997), such as inflammatory disease, infections with viruses, bacteria, and parasites, malignant diseases, autoimmune diseases, and rejection episodes following organ transplantation (www.neopterin.net/neopterin_e.pdf).

Neopterin has been described as a marker for cardiovascular risk and a possible pathogenic factor in atherosclerosis (Avanzas et al., Drug News & Perspectives 22(4): 215 (2009); see, also, Forsblad et al., Int'l Angiology 21(2): 173-179 (2002), and Tatzber et al., Atherosclerosis 89(2): 203-208 (August 1991); see, also, Fuchs et al., Curr. Med. Chem. 16(35): 4644-4653 (2009); Avanzas et al., Clin. Chem. 55(6): 1056-1057 (2009); Ariyarajah, South. Med. J. 101(5): 461-463 (May 2008); Kaski et al., Clin. Chem. 51: 1902-1903 (2005); Kaski et al., JACC 42(6): 1142-1143 (Sep. 17, 2003); and U.S. Pat. App. Pub. No. 2010/0159474). Neopterin has been described as an independent predictor of all-cause and cardiovascular mortality in individuals with and without stable coronary artery disease (Grammer et al., Clin. Chem. 55(6): 1135-1146 (2009)). Elevated plasma levels of neopterin are considered to have prognostic value in patients with stable coronary artery disease by identifying patients at long-term risk of death or recurrent acute coronary events after acute coronary syndromes (Ray et al., Circulation 115: 3071-3078 (2007)). Serum neopterin levels reportedly may indicate future plaque instability in stable angina patients and long-term risk of death or recurrent acute coronary events after myocardial infarction in ST-elevation myocardial infarction patients (Djordjevic et al., Clin. Chem. and Lab. Med. 46(8): 1149-1155 (2008)). Circulating levels of neopterin are elevated in patients with complex coronary lesions in unstable angina (or unstable angina pectoris) (see, e.g., Garcia-Moll et al., J. Amer. Coll. Cardiol. 35: 956-962 (2000)). Elevated plasma levels of neopterin also have been described in patients with chronic stable angina (CSA) (Estevez-Loureiro et al., Atherosclerosis 207(2): 514-518 (2009)) and patients with chronic stable angina pectoris having carotid plaques of complex morphology (Sugioka et al., Atherosclerosis 208: 524-530 (2010)). Elevated plasma levels of neopterin are considered to be predictive of left ventricular dysfunction in patients with CSA (Estevez-Loureiro et al. (2009), supra). Immunohistochemical staining of the complex carotid plaques reportedly revealed abundant neopterin-positive macrophages (Sugioka et al. (2010), supra). Thus, neopterin can be considered an important biomarker of plaque destabilization in carotid artery atherosclerotic lesions in patients with stable angina pectoris (see, also, Adachi et al., Heart 93: 1537-1541 (2007), regarding plaque destabilization in coronary atherosclerotic lesions, and Zouridakis et al., Circulation 110: 1747-1753 (2004), regarding rapid coronary artery disease progression in patients with stable angina pectoris) and major adverse coronary events in patients with chronic stable angina pectoris (Avanzas et al., European Heart J. 26: 457-463 (2005)). Neopterin is also a predictor of left ventricular remodeling (LVR) in patients with coronary artery disease. A correlation between an elevation in the level of neopterin and LVR in patients with ST-segment elevation myocardial infarction (STEMI) also has been described (Dominguez-Rodriguez et al., Atherosclerosis 211(2): 574-578 (August 2010)). High neopterin levels in patients with STEMI undergoing primary percutaneous coronary intervention were predictive of LVR one year later (Dominguez-Rodriguez et al. (2010), supra). Elevated serum levels of neopterin also have been described in patients with non-rheumatic aortic valve stenosis (Naito et al., Int'l J. Cardiol. (2010), doi: 10.1016/j.ijcard.2010.02.035). Elevated serum levels of neopterin (and independently C-reactive protein (CRP)) have been described as predictive of fatal ischemic heart disease in diabetic patients (Vengen et al., Atherosclerosis 207(1): 239-244 (November 2009)). Elevated levels of neopterin also have been described as associated with the severity of coronary artery disease (CAD) (Alber et al., Int'l J. of Cardiology 135(1): 27-35 (Jun. 12, 2009)). It has been proposed that the association of elevated levels of neopterin and the severity of CAD might be useful in identifying patients eligible for revascularization procedures (Alber et al. (2009), supra). Patient with isolated coronary artery ectasia have been described as having elevated levels of neopterin compared to patients with normal coronary arteries (Sahin et al., South. Med. J. 101(5): 476-479 (May 2008)). Serum neopterin concentrations reportedly have a high correlation with thrombolysis in myocardial infarction (TIMI) risk scores and may represent a useful marker in stratifying patients with acute coronary syndromes (Johnston et al., Coronary Artery Disease 17: 511-516 (2006)).

Increased neopterin levels also can be indicative of acute viral infections and other infections (see, e.g., U.S. Pat. App. Pub. No. 2009/0104602 regarding use of neopterin with other marker(s) in diagnosis of tuberculosis). Screening for elevated neopterin levels in blood donation reduces the risk of the spread of infections. Such screening is mandated in Austria. Cytomegalovirus (CMV) infection reportedly is significantly more prevalent in blood donors with serum neopterin levels above 10 nmol/L (Honlinger et al., Dtsch. Med. Wochenschr. 114: 172-176 (1989)). Acute CMV infections among blood donors reportedly presented with elevated serum neopterin levels even before CMV IgG/IgM antibodies were detected (Schennach et al., Med. Micro. Immunol. 191(2): 115-118 (2002)). Neopterin and albumin levels in serum and cerebrospinal fluid (CSF) have been reported to correlate with HIV-1 RNA levels in CSF (Andersson et al., J. Neurovirol. 7(6): 542-547 (December 2001); see, also, Hagberg et al., AIDS Res. Ther. 7: 15 (2010), and Wirleitner et al., Molec. Immunol. 42(2): 183-194 (February 2005)). In this regard, neopterin has been identified as an inexpensive and reliably measured serum marker for monitoring patients with advanced HIV-1 infection, particularly in resource-limited settings (Mildvan et al., Clin. Infect. Dis. 40: 853-858 (2005)), and urine levels of neopterin have been described as useful in predicting survival in HIV-positive patients (Rogstad et al., Int'l J. STD & AIDS 9: 326-329 (1998); see, also, Fuchs et al., Clin. Chem. 35: 1746-1749 (1989)). In contrast, saliva levels of neopterin reportedly do not correlate significantly with HIV-1 infection (Evans et al., Clin. Chem. 41(6): 950-951 (1995)). Neopterin screening of blood donors led to the discovery of an HBsAg-positive donor and a donor with adenovirus infection (Fisenk et al., Scand. J. Infect. Dis. 37(8): 599-604 (2005)). Increased neopterin levels also have been reported in asymptomatic blood donors with human parvovirus B19 infection (Schennach et al., J. Infect. Dis. 186: 1494-1497 (2002)).

Malignancy also can be associated with elevated neopterin levels. Neopterin levels in bodily fluids like urine, serum, plasma, and ascites reportedly parallel the course of the disease, and a higher level of neopterin is considered to be an independent predictor of a shorter survival period (Sucher et al., Cancer Letters 287(1): 13-22 (Jan. 1, 2010). Serum neopterin levels reportedly are elevated in patients with advanced gastric cancer and correlated with prognostic parameters and overall survival (Unal et al., J. Invest. Surgery 22(6): 419-425 (December 2009)). The presence of two or more comorbid conditions reportedly was associated with a significant increase in neopterin levels in urine of patients with breast carcinoma (Melcharova et al., Eur. J. Cancer Care 19(3): 340-345 (May 2010)). Urinary neopterin reportedly increases in most patients with epithelial ovarian carcinoma and is considered to be an independent prognostic indicator (Melichar et al., Pteridines 17: 145-153 (2006); see, also, Melichar et al., Int'l J. Gyn. Cancer 16(1): 240-252 (January 2006); and U.S. Pat. App. Pub. No. 2004/0180387). Increased urinary neopterin was associated with toxicity with chemotherapeutic treatment with paclitaxel/platinum (Melichar et al. (2006), supra). Elevated pre-operative neopterin has been described as a reliable prognostic indicator of lower survival probability for lung cancer (Prommegger et al., The Annals of Thor. Surgery 70(6): 1861-1864 (December 2000)) and breast cancer (Kocer et al., Central European J. of Med. DOI: 10.2478/s11536-0,0-0017-6 (2009)).

Autoimmunity also can be associated with elevated neopterin levels. Urinary neopterin is considered to be a potentially useful marker for monitoring disease activity in patients with systemic lupus erythematosus (Leohirun et al., Clin. Chem. 37: 47-50 (1991)). Patients with rheumatoid arthritis have been reported to have higher levels of neopterin in synovial fluid and urine than patients with osteoarthritis (Hagihara et al., Clin. Chem. 36(4): 705-706 (1990); Krause et al., Ann. Rheum. Dis. 48: 636-640 (1989)); and Reibnegger et al., Arthritis & Rheumatism 29(9): 1063-1070 (September 1986)).

Neopterin also has been described as a marker for transplants. Neopterin reportedly is excreted at high levels during allograft rejection and is considered to be a marker for the detection of acute rejection after heart transplantation (Havel et al., J. Heart Transplant 8(2): 167-170 (March-April 1989)). Neopterin also can be a marker for the early diagnosis of renal allograft rejection as well as poorer long-term graft survival (Reibnegger et al., Transplantation 52: 58-63 (1991); see, also, Chin et al., Clin. & Exp. Immunol. 152(2): 239-244 (May 2008)); Carlson, Clin. Lab Med. 12(1): 99-111 (March 1992); and Lee et al., J. Formos Med. Assoc. 91: 1209-1212 (1992)). Measurement of elevated levels of neopterin in bile fluid and urine is proposed to distinguish liver allograft rejection from infectious disease in transplant patients, whereas measurement of decreased levels of neopterin after anti-rejection therapy reportedly evidences successful treatment (Hausen et al., Clin. Chem. 39: 45-47 (1993); see, also, Margreiter et al., Transplant Int. 5[Suppl 1]: S199-S200 (1992)). Amyloid A, in combination with urinary neopterin and urinary amylase, enabled differential diagnosis between rejection, bacterial infection, and viral infection after simultaneous pancreas and kidney transplantation (Muller et al., Transplant Int'l 10(3): 185-191 (1997)).

Since neopterin is a stable molecule, it can be assayed in protein-containing bodily fluids, such as serum, plasma, cerebrospinal fluid, pancreatic juice or ascites, by radioimmunoassay, albeit with its associated radiological hazards and regulatory issue, and, only very recently, by competitive enzyme-linked immunosorbent assay (ELISA) using horseradish peroxidase (HRPO)-labeled neopterin. Neopterin also can be assayed in urine by high pressure liquid chromatography (HPLC; see, e.g., Huber et al., J. Chromatography B: Biomed. Sci. App. 666(2): 223-232 (April 1995) regarding HPLC of neopterin in serum) with fluorescence detection after appropriate sample clean-up.

In some EU countries testing of neopterin to detect cellular immune activation has been mandatory since 1995 (Bayer et al., Clin. Lab. 51: 495-504 (2005)); commercial assays from IBL (Minneapolis, Minn., and Hamburg, Germany; see, also, Bayer et al. (2005), supra; and Westermann et al., Clin. Chem. Lab. Med. 38(4): 345-353 (2000)), BRAHMS Diagnostics GmbH (Berlin, Germany; see, e.g., U.S. Pat. No. 5,698,408), and the newest from Siemens (Dade-Behring) are in use. The assay available from BRAHMS comprises a microplate with sheep polyclonal anti-neopterin/neopterin alkaline phosphatase conjugate and requires about two hours and 30 minutes to run. The assay available from IBL comprises a microplate with goat anti-rabbit/rabbit anti-neopterin/neopterin horseradish peroxidase conjugate and requires one hour and 40 minutes to run. An alternative embodiment of the assay available from IBL comprises a microplate with goat anti-mouse/murine monoclonal anti-neopterin/neopterin horseradish peroxidase conjugate and requires 1 hour and 45 minutes to run. The lattermost claims great improvements over the former, the improvements being analysis time (two hours) and sample volume (10 μL). An agent for immunoassay of neopterin comprising an anti-neopterin antibody and an oxidizing agent is described in U.S. Pat. No. 5,439,799.

In view of the foregoing, the present disclosure seeks to provide materials and methods for immunoassay of neopterin that offer advantages over currently available materials and methods. This and other objects of the present disclosure, as well as inventive features, will become apparent from the detailed description provided herein.

### SUMMARY

A method of determining the presence, amount or concentration of a pterin in a test sample is provided. The method comprises assaying the test sample for a pterin by an immunoassay employing as a capture agent a pterin of formula I or II:

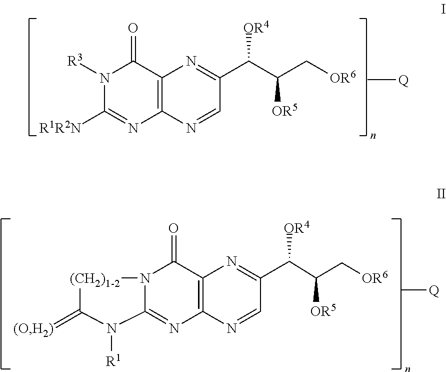

wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido

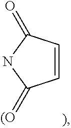

mercapto (SH) and carboxyl (CO₂H), conjugated to Q, wherein Q is a solid support, and wherein “n” is 1-20. The immunoassay employs a detectably labeled anti-pterin antibody, such as an anti-pterin antibody labeled with an acridinium compound. The acridinium compound can be an acridinium-9-carboxamide, e.g., an acridinium-9-carboxamide of formula III:

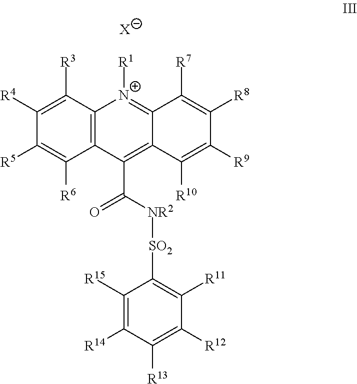

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion, or an acridinium-9-carboxylate aryl ester, e.g., an acridinium-9-carboxylate aryl ester of formula IV:

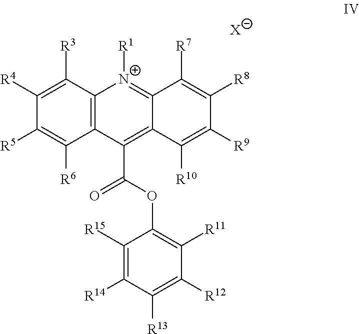

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

A method of determining the presence, amount or concentration of neopterin in a test sample is also provided. The method comprises assaying the test sample for neopterin by a chemiluminescent microparticle immunoassay employing an anti-neopterin antibody as a capture agent. The immunoassay can employ labeled neopterin or a labeled anti-neopterin antibody, wherein the label is an acridinium compound, such as an acridinium-9-carboxamide, e.g., an acridinium-9-carboxamide of formula III as described above, or an acridinium-9-carboxylate aryl ester, e.g., an acridinium-9-carboxylate aryl ester of formula IV as described above.

Yet another method of determining the presence, amount or concentration of neopterin in a test sample is provided. The method comprises assaying the test sample for neopterin by an immunoassay employing as a conjugate an anti-neopterin antibody labeled with an acridinium compound. The immunoassay can be a chemiluminescent microparticle immunoassay.

Still yet another method of determining the presence, amount or concentration of neopterin in a test sample is provided. The method comprises assaying the test sample for neopterin by an immunoassay employing as a tracer neopterin labeled with an acridinium compound, e.g., compound 10a, 10b, or 10c in FIG. 2, compound 25 in FIG. 7, or compound 30a, 30b, 30c, 31a, 31b, 31c, 32a, 32b, or 32c in FIG. 9. The immunoassay can be a chemiluminescent microparticle immunoassay.

With regard to the above methods, the test sample can be plasma or serum. The test sample can be from a patient, and the method can further comprise diagnosing, prognosticating, or assessing the efficacy of therapeutic/prophylactic treatment of a condition comprising inflammation in the patient. If the method further comprises assessing the efficacy of therapeutic/prophylactic treatment of the patient, the method optionally further comprises modifying the therapeutic/prophylactic treatment of the patient as needed to improve efficacy. The method can further comprise assaying, simultaneously or sequentially, in either order, by immunoassay, e.g., chemiluminescent microparticle immunoassay, or other assay, another marker selected from the group consisting of myeloperoxidase (MPO), neutrophil gelatinase-associated lipocalin (NGAL), C-reactive protein (CRP), and calcitonin. The method can be adapted for use in an automated system or a semi-automated system.

Also provided is an anti-neopterin antibody labeled with an acridinium compound, such as an acridinium-9-carboxamide, e.g., an acridinium-9-carboxamide of formula III as described above, or an acridinium-9-carboxylate aryl ester, e.g., an acridinium-9-carboxylate aryl ester of formula IV as described above.

A conjugate/complex comprising an anti-neopterin antibody and a carrier scaffold, wherein the ratio of antibody:carrier scaffold is greater than about 4, is also provided. The carrier scaffold is selected from the group consisting of a protein, a polysaccharide, a polynucleotide, dextran, streptavidin, and a dendrimer. The anti-neopterin antibody is optionally labeled.

Further provided is a pterin. The pterin has the formula I or II:

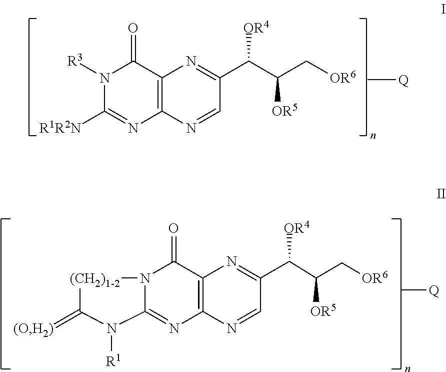

wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido

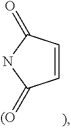

mercapto (SH) and carboxyl (CO₂H), conjugated to Q, wherein Q is a solid support, a protein, or a detectable label, and wherein “n” is 1-20. The detectable label can be an acridinium compound. The pterin can be neopterin, such as neopterin labeled with an acridinium compound. The acridinium compound can be an acridinium-9-carboxamide, e.g., an acridinium-9-carboxamide of formula III as described above, in which case the pterin labeled with an acridinium compound can be compound 10a, 10b, or 10c in FIG. 2, compound 25 in FIG. 7, or compound 30a, 30b, 30c, 31a, 31b, 31c, 32a, 32b, or 32c in FIG. 9. Alternatively, the acridinium compound can be an acridinium-9-carboxylate aryl ester, e.g., an acridinium-9-carboxylate aryl ester of formula IV as described above.

Still further provided is a conjugate comprising (i) a pterin labeled with an acridinium compound as described above and (ii) a carrier scaffold. The carrier scaffold can be selected from the group consisting of a protein, a polysaccharide, a polynucleotide, dextran, streptavidin, and a dendrimer, wherein the ratio of pterin:label is greater than about 10.

Even still further provided is an immunogen comprising neopterin and a carrier protein, wherein the neopterin is directly conjugated to the carrier protein. The carrier protein can be bovine serum albumin (BSA), keyhole limpet hemocyanin (KLH), or thryroglobulin (TG).

A conjugate comprising the above-described immunogen and an acridinium compound is also provided. The acridinium compound can be an acridinium-9-carboxamide.

Also provided is an immunogen comprising a carrier protein and 2-N-(5-carboxypentyl)-D-neopterin, 2-N-(3-aminopropyl)-D-neopterin, 2-N-(2-carboxyethyl)-D-neopterin, 3-N-(2-carboxyethyl)-D-neopterin, or 2-N-(2-carboxyethyl)-2,3-N,N′-(1-oxopropylidinyl)-D-neopterin. The carrier protein can be BSA, KLH or TG.

Further provided is a conjugate comprising the above-described immunogen and an acridinium compound. The acridinium compound can be an acridinium-9-carboxamide.

Still further provided is a kit for assaying a test sample for a pterin. The kit comprises (i) a pterin of formula I or II (as described above) conjugated to Q, wherein Q is a solid support, as a capture agent and (ii) instructions for assaying the test sample for a pterin by immunoassay.

Even still further provided is a kit for assaying a test sample for neopterin. The kit comprises (i) an anti-neopterin antibody as a capture agent and (ii) instructions for assaying the test sample for neopterin by chemiluminescent microparticle immunoassay.

Another kit for assaying a test sample for neopterin is provided. The kit comprises (i) an anti-neopterin antibody labeled with an acridinium compound as a conjugate and (ii) instructions for assaying the test sample for neopterin by immunoassay.

Yet another kit for assaying a test sample for neopterin is provided. The kit comprises (i) neopterin labeled with an acridinium compound as a tracer and (ii) instructions for assaying the test sample for neopterin by immunoassay.

### BRIEF DESCRIPTION OF FIGURES

FIG. 1 is a graph of R/R₀ vs. concentration (nM) of neopterin ([Neopterin]) comparing two different anti-neopterin antibodies, wherein -♦- represents a mouse anti-neopterin antibody available from IBL-America, Minneapolis, Minn. (IBL IgG) and -▪- represents a mouse anti-neopterin antibody available from Antibodies-online GmbH, Atlanta, Ga. (117-14E 10 IgG).

FIG. 2 is a graph comparing the receiver operating curves (ROC) for neopterin, myeloperoxidase and C-reactive protein, wherein the solid line represents neopterin (AUC=0.718; cutoff 7.9), the dashed line represents myeloperoxidase (MPO) (AUC=0.555; cutoff 179), and the dotted lines represents C-reactive protein (hsCRP) (AUC=0.611; cutoff 13.9).

### DETAILED DESCRIPTION

The present disclosure provides methods of assaying for (i) a pterin by immunoassay employing a pterin as a capture agent, (ii) neopterin by chemiluminescent microparticle immunoassay employing an anti-neopterin antibody as a capture agent, (iii) neopterin by an immunoassay employing an acridinium-labeled anti-neopterin antibody as a conjugate, and (iv) neopterin by an immunoassay employing acridinium-labeled neopterin as a tracer. Such methods enable decreased assay time, decreased sample volume, and ease of manufacture. Such methods also increase resolution of combinations of markers, such as neopterin and one or more other inflammatory markers, and enable better monitoring of patient response to anti-inflammatory treatment, such as with anti-tumor necrosis factor α (TNF-α) biologics, such as Humira (Abbott Laboratories, Abbott Park, Ill.). Also provided is an acridinium-labeled anti-neopterin antibody, a conjugate/complex comprising an anti-neopterin antibody and a carrier scaffold at a ratio greater than about 4, a pterin conjugated to a solid support, a protein or a detectable label, a conjugate comprising an acridinium-labeled pterin and a carrier scaffold, an immunogen comprising neopterin directly conjugated to a carrier protein, a conjugate comprising such an immunogen and an acridinium compound, an immunogen comprising a carrier protein and a neopterin hapten, and a conjugate comprising such an immunogen and an acridinium compound. A kit for assaying a pterin comprising a pterin as a capture agent and instructions for immunoassay, a kit for assaying neopterin comprising an anti-neopterin antibody as a capture agent and instructions for chemiluminescent microparticle immunoassay, a kit for assaying neopterin comprising an acridinium-labeled anti-neopterin antibody as a conjugate and instructions for immunoassay, and a kit for assaying neopterin comprising acridinium-labeled neopterin as a tracer and instructions for immunoassay are also provided.

### DEFINITIONS

The following terms are relevant to the present disclosure:

“About” refers to approximately a +/−10% variation from the stated value. It is to be understood that such a variation is always included in any given value provided herein, whether or not specific reference is made to it.

“Acyl” means RC(O)—.

“Alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

“Alkoxy” or “alkoxyl” means an alkyl group, as defined herein, appended to the parent molecular moiety through an oxygen atom, representative examples of which include, but are not limited to, methoxy, ethoxy, propoxy, 2-propoxy, butoxy, tert-butoxy, pentyloxy, and hexyloxy.

“Alkyl” means a straight or branched chain hydrocarbon containing from 1 to 10 carbon atoms, which is optionally substituted. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

“Alkylcarbonyl” means an alkyl group attached to the parent molecular moiety through a carbonyl group.

“Alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

“Amido” means —C(O)NRₐR_{b}, wherein Rₐ and R_{b} are independently selected from the group consisting of hydrogen and alkyl.

“Amino” means —NRₐR_{b}, wherein Rₐ and R_{b} are independently selected from the group consisting of hydrogen, alkyl and alkylcarbonyl.

“Anion” refers to an anion of an inorganic or organic acid. Examples include, but are not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid, fluorosulfonic acid, and any combinations thereof.

“Antibody” and “antibodies” refer to monoclonal antibodies, polyclonal antibodies, multispecific antibodies, human antibodies, humanized antibodies (fully or partially humanized), animal antibodies (such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)SH fragments, F(ab′)₂ fragments, Fd fragments, Fv fragments, single chain Fv fragments (“scFv”), disulfide-linked Fvs (“sdFv”), single-chain polypeptides containing only one light chain variable domain, single-chain polypeptides containing the three complementarity determining regions (CDRs) of the light-chain variable domain, single-chain polypeptides containing only one heavy chain variable region, single-chain polypeptides containing the three CDRs of the heavy chain variable region, anti-idiotypic (“anti-Id”) antibodies, diabodies, dual-domain antibodies, dual variable domain (DVD) or triple variable domain (TVD) antibodies (dual-variable domain immunoglobulins and methods for making them are described in Wu, C., et al., Nature Biotechnology, 25(11): 1290-1297 (2007), and International Pat. App. Pub. No. WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA and IgY), class (for example, IgG₁, IgG₂, IgG₃, IgG₄, IgA₁ and IgA₂) or subclass. An antibody, whose affinity (namely, K_{D}, k_{d} or kₐ) has been increased or improved via the screening of a combinatory antibody library that has been prepared using bio-display, is referred to as an “affinity maturated antibody.” For simplicity sake, an antibody against an analyte is frequently referred to herein as being either an “anti-analyte antibody” or merely an “analyte antibody” (e.g., an anti-neopterin antibody or a neopterin antibody).

“Aryalkyl” means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

“Aryl” means a phenyl group, or a bicyclic or tricyclic fused ring system in which one or more of the fused rings is a phenyl group. Bicyclic fused ring systems are exemplified by a phenyl group fused to a cycloalkenyl group, as defined herein, a cycloalkyl group, as defined herein, or another phenyl group. Tricyclic fused ring systems are exemplified by a bicyclic fused ring system fused to a cycloalkenyl group, as defined herein, a cycloalkyl group, as defined herein, or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkoxy, alkyl, carboxyl, halo, and hydroxyl.

“Autoimmune disease” refers to the loss of immunological tolerance to self antigens. Some criteria for a diagnosis of autoimmune disease include: (1) the presence of circulating autoantibodies; (2) autoantibodies observed in the affected organ; (3) target antigen identified; (4) inducible in an animal model either by immunization with antigen, serum, or autoantibody transfer; and (5) responsive to immunosuppressive therapy or immunoabsorption. Other characteristics of autoimmune disease include its: (a) increased prevalence in women; (b) familial clustering (although this varies with disease); (c) asymptomatic risk (i.e., the presence of autoantibodies may precede the disease by years); (d) periodic nature; and (e) chronic nature.

“Carboxy” or “carboxyl” refers to —CO₂H.

“Carboxyalkyl” refers to an alkyl group that is substituted with one or more carboxy groups.

“Cardiovascular disease” refers to various clinical diseases, disorders or conditions involving the heart, blood vessels or circulation. The diseases, disorders or conditions can be due to atherosclerotic impairment of coronary, cerebral or peripheral arteries. Cardiovascular disease includes, but is not limited to, coronary artery disease, peripheral vascular disease, atherosclerosis, hypertension, myocardial infarction (i.e., heart attack, e.g., primary or secondary, which occurs when an area of heart muscle dies or is damaged because of an inadequate supply of oxygen to that area), myocarditis, acute coronary syndrome, angina pectoris (i.e., chest discomfort caused by inadequate blood flow through the blood vessels (coronary vessels) of the myocardium), sudden cardiac death, cerebral infarction, restenosis, syncope, ischemia, transient ischemic attack, reperfusion injury, vascular occlusion, carotid obstructive disease, cardiovascular autoimmune disease, etc. By “cardiovascular autoimmune disease” is meant any deviation from a healthy or normal condition of the heart that is due to an underlying autoimmune disease, including any structural or functional abnormality of the heart, or of the blood vessels supplying the heart, that impairs typical functioning. Examples of cardiovascular autoimmune diseases include myocarditis, cardiomyopathy, and ischemic heart disease, each due to an underlying autoimmune disease. “Myocarditis” refers to inflammation of the myocardium. Myocarditis can be caused by a variety of conditions, such as viral infection, sarcoidosis, rheumatic fever, autoimmune diseases (such as systemic lupus erythematosus, etc.), and pregnancy. “Cardiomyopathy” refers to a weakening of the heart muscle or a change in heart muscle structure. It is often associated with inadequate heart pumping or other heart function abnormalities. Cardiomyopathy can be caused by viral infections, heart attacks, alcoholism, long-term, severe high blood pressure, nutritional deficiencies (particularly selenium, thiamine, and L-carnitine), systemic lupus erythematosus, celiac disease, and end-stage kidney disease. Types of cardiomyopathy include dilated cardiomyopathy, hypertrophic cardiomyopathy, and restrictive cardiomyopathy. “Dilated cardiomyopathy” refers to a global, usually idiopathic, myocardial disorder characterized by a marked enlargement and inadequate function of the left ventricle. Dilated cardiomyopathy includes ischemic cardiomyopathy, idiopathic cardiomyopathy, hypertensive cardiomyopathy, infectious cardiomyopathy, alcoholic cardiomyopathy, toxic cardiomyopathy, and peripartum cardiomyopathy. “Hypertrophic cardiomyopathy” refers to a condition resulting from the right and left heart muscles growing to be different sizes. “Restrictive cardiomyopathy” refers to a condition characterized by the heart muscle's inability to relax between contractions, which prevents it from filling sufficiently. “Ischemic heart disease” refers to any condition in which heart muscle is damaged or works inefficiently because of an absence or relative deficiency of its blood supply; most often caused by atherosclerosis, it includes angina pectoris, acute myocardial infarction, and chronic ischemic heart disease.

“Component,” “components,” and “at least one component,” refer generally to a capture antibody, a detection or conjugate antibody, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

“Control” refers to a composition known to not contain an analyte (“negative control”), such as a pterin, e.g., neopterin, or an anti-pterin antibody, e.g., an anti-neopterin antibody, or to contain an analyte (“positive control”). A positive control can comprise a known concentration of an analyte, such as a pterin, e.g., neopterin, or an anti-pterin antibody, e.g., an anti-neopterin antibody. “Control,” “positive control,” and “calibrator” may be used interchangeably herein to refer to a composition comprising a known concentration of an analyte. A “positive control” can be used to establish assay performance characteristics and is a useful indicator of the integrity of reagents (e.g., analytes).

“Cyano” means a —CN group.

“Cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

“Cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

“Epitope,” “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Halide” means a binary compound, of which one part is a halogen atom and the other part is an element or radical that is less electronegative than the halogen, e.g., an alkyl radical.

“Halogen” means —Cl, —Br, —I or —F.

“Hydrogen peroxide-generating enzyme” refers to an enzyme that can generate hydrogen peroxide. Examples of hydrogen peroxide-generating enzymes are listed below in Table 1.

TABLE 1

| | IUBMB Enzyme | |
|---|---|---|
| Common Name | Nomenclature | Preferred Substrate |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| Aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary |
| | | alcohol |
| | | (2-naphthyl)methanol |
| | | 3-methoxybenzyl |
| | | alcohol |
| aryl-aldehyde oxidase | EC 1.2.3.9 | an aromatic aldehyde |
| Catechol oxidase | EC 1.1.3.14 | Catechol |
| Cholesterol oxidase | EC 1.1.3.6 | Cholesterol |
| Choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| Ecdysone oxidase | EC 1.1.3.16 | Ecdysone |
| ethanolamine oxidase | EC 1.4.3.8 | Ethanolamine |
| Galactose oxidase | EC 1.1.3.9 | D-galactose |
| Glucose oxidase | EC 1.1.3.4 | β-D-glucose |
| Glutathione oxidase | EC 1.8.3.3 | Glutathione |
| glycerol-3-phosphate oxidase | EC 1.1.3.21 | sn-glycerol 3-phosphate |
| Glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| Nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| oxalate oxidase | EC 1.2.3.4 | Oxalate |
| polyamine oxidase | EC 1.5.3.11 | 1-N-acetylspermine |
| Polyphenol oxidase | EC 1.14.18.1 | |
| Polyvinyl-alcohol oxidase | EC 1.1.3.30 | polyvinyl alcohol |
| prenylcysteine oxidase | EC 1.8.3.5 | an S-prenyl-L-cysteine |
| Protein-lysine 6-oxidase | EC 1.4.3.13 | peptidyl-L-lysyl-peptide |
| putrescine oxidase | EC 1.4.3.10 | butane-1,4-diamine |
| Pyranose oxidase | EC 1.1.3.10 | D-glucose |
| | | D-xylose |
| | | L-sorbose |
| | | D-glucono-1,5-lactone |
| Pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8- |
| | | dioxo-1,2,3,4,5,6,7,8- |
| | | octahydroquinoline-2,4- |
| | | dicarboxylate |
| Pyruvate oxidase | EC 1.2.3.3 | Pyruvate |
| Pyruvate oxidase (CoA-acetylating) | EC 1.2.3.6 | Pyruvate |
| Reticuline oxidase | EC 1.21.3.3 | Reticuline |
| retinal oxidase | EC 1.2.3.11 | Retinal |
| Rifamycin-B oxidase | EC 1.10.3.6 | rifamycin-B |
| Sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| Thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| Vanillyl-alcohol oxidase | EC 1.1.3.38 | vanillyl alcohol |
| Xanthine oxidase | EC 1.17.3.2 | Xanthine |
| xylitol oxidase | EC 1.1.3.41 | Xylitol |

“Hydroxyl” means an —OH group.

“Label” and “detectable label” mean a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

“Neopterin” is a derivative of pteridine and a byproduct of the guanosine triphosphate-biopterin pathway. Levels of neopterin increase during monocyte/macrophage activation and inflammation. Neopterin has the chemical structure:

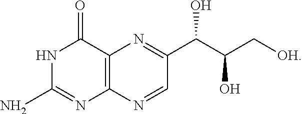

Neopterin is also known as D-erthryo-neopterin and 2-amino-6-[(1S,2R)-1,2,3-trihydroxypropyl]-4(3H)pteridinone. It has the formula C₉H₁₁N₅O₄, a molecular weight of 253.21, and the CAS Reg. No. [2009-64-5].

“Nitro” means a —NO₂ group.

“Oxoalkyl” refers to an alkyl group that is substituted with one or more oxy groups.

“Patient” and “subject” may be used interchangeably herein to refer to an animal, such as a bird (e.g., a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, and a mouse) and a primate (for example, a monkey, a chimpanzee, and a human). Preferably, the patient or subject is a human, such as a human at risk for or having a condition comprising inflammation.

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). The present disclosure provides exemplary predetermined levels. However, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, the correlations as described herein should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., a pterin, such as neopterin, or an anti-pterin antibody, such as an anti-neopterin antibody) entails release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte.

“Pterin” is a compound of formula I or II:

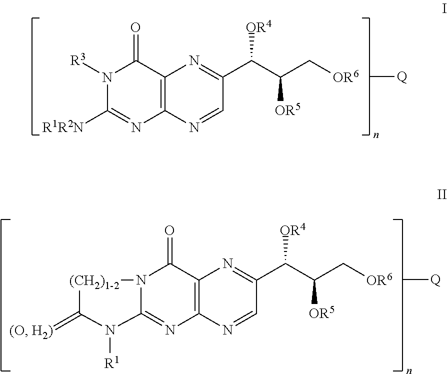

wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido

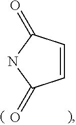

mercapto (SH) and carboxyl (CO₂H), optionally conjugated to Q, wherein Q is a detectable label, a protein, or a solid support, and wherein “n” is 1-20.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, dual-variable domain immunoglobulins (DVD-Ig®s; Wu, C., et al., Nature Biotechnology, 25:1290-1297 (2007)), and other antibodies as described herein above. The term “bifunctional antibody,” as used herein, refers to an antibody that comprises a first arm having a specificity for one antigenic site and a second arm having a specificity for a different antigenic site, i.e., the bifunctional antibodies have a dual specificity.

“Risk” refers to the possibility or probability of a particular event occurring either presently, or, at some point in the future. “Risk stratification” or “prognosticating the risk” refers to an array of known clinical risk factors that allows physicians to classify patients into a low, moderate, high or highest risk of developing a particular disease, disorder or condition.

“Sample,” “test sample,” and “patient sample” may be used interchangeably herein. The sample, such as a sample of urine, serum, plasma, amniotic fluid, cerebrospinal fluid, placental cells or tissue, endothelial cells, leukocytes, or monocytes, can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

Neopterin concentrations in normal serum and plasma do not differ (5.2±2.5 nmol/L), and a sample volume of 20-100 μL of serum, plasma or cerebrospinal fluid is sufficient for a single immunoassay. The content of neopterin in serum or plasma is stable for three days at room temperature. Cooling at 4° C. is adequate for storage up to one week. Samples can be kept up to about three months if kept frozen (−20° C.). Repeated thawing-freezing cycles must be avoided. Bile fluid samples preferably are diluted in physiological saline solution.

“Series of calibrating compositions” refers to a plurality of compositions comprising a known concentration of an analyte, e.g., a pterin (including any metabolites thereof or cross-reacting substances), such as neopterin, or an anti-pterin antibody, such as an anti-neopterin antibody, wherein each of the compositions differs from the other compositions in the series by the concentration of the analyte.

“Solid phase” refers to any material that is insoluble, or can be made insoluble by a subsequent reaction. The solid phase can be chosen for its intrinsic ability to attract and immobilize a capture agent. Alternatively, the solid phase can have affixed thereto a linking agent that has the ability to attract and immobilize the capture agent. The linking agent can, for example, include a charged substance that is oppositely charged with respect to the capture agent itself or to a charged substance conjugated to the capture agent. In general, the linking agent can be any binding partner (preferably specific) that is immobilized on (attached to) the solid phase and that has the ability to immobilize the capture agent through a binding reaction. The linking agent enables the indirect binding of the capture agent to a solid phase material before the performance of the assay or during the performance of the assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

“Specific” and “specificity” in the context of an interaction between members of a specific binding pair (e.g., an antigen (or a fragment thereof) and an antibody (or antigenically reactive fragment thereof)) refer to the selective reactivity of the interaction. The phrase “specifically binds to” and analogous phrases refer to the ability of antibodies (or antigenically reactive fragments thereof) to bind specifically to an antigen, e.g. a pterin, such as neopterin (or a fragment thereof), and not bind specifically to other antigens (or fragments thereof).

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Sulfo” means SO₃H.

“Sulfoalkyl” refers to an alkyl group to which a sulfonate group is bonded, wherein the alkyl is bonded to the molecule of interest.

“Tracer” means an analyte or analyte fragment conjugated to a label, such as a pterin, e.g., neopterin, conjugated to a fluorescein moiety, wherein the analyte conjugated to the label can effectively compete with the analyte for sites on an antibody specific for the analyte.

“Variant” as used herein means a polypeptide that differs from a given polypeptide (e.g., an anti-pterin antibody, such as an anti-neopterin antibody, or a pterin, such as neopterin) in amino acid sequence by the insertion, deletion, or conservative substitution of amino acids, but that retains the biological activity of the given polypeptide (e.g., can compete with anti-neopterin antibody as defined herein for binding to neopterin or can compete with neopterin for binding to anti-neopterin antibody). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol. 157: 105-132 (1982)). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101, which is incorporated herein by reference). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-pterin antibody, such as an anti-neopterin antibody, that differs from the corresponding fragment of an anti-pterin antibody, such as an anti-neopterin antibody, in amino acid sequence but is still antigenically reactive and can compete with the corresponding fragment of anti-pterin antibody, such as an anti-neopterin antibody, for binding to a pterin, such as neopterin. Similarly, “variant” also can be used to refer to a fragment, such as an immunologically reactive fragment, of a pterin, such as neopterin, that differs from the corresponding fragment of a pterin, such as neopterin, in amino acid sequence but is still immunologically reactive and can compete with the corresponding fragment of a pterin, such as neopterin, for binding to an anti-pterin antibody, such as an anti-neopterin antibody. “Variant” also can be used to describe a polypeptide or a fragment thereof that has been differentially processed, such as by proteolysis, phosphorylation, or other post-translational modification, yet retains its antigenic reactivity, e.g., ability of an anti-pterin antibody, such as an anti-neopterin antibody, to bind to a pterin, such as neopterin, or immunological reactivity, e.g, ability of a pterin, such as neopterin, to bind to an anti-pterin antibody, such as an anti-neopterin antibody.

The terminology used herein is for the purpose of describing particular embodiments only and is not otherwise intended to be limiting.

### Method of Determining the Presence, Amount or Concentration of an Analyte in a Test Sample

A method of determining the presence, amount or concentration of a pterin in a test sample is provided. The method comprises assaying the test sample for a pterin by an immunoassay employing as a capture agent a pterin of formula I or II:

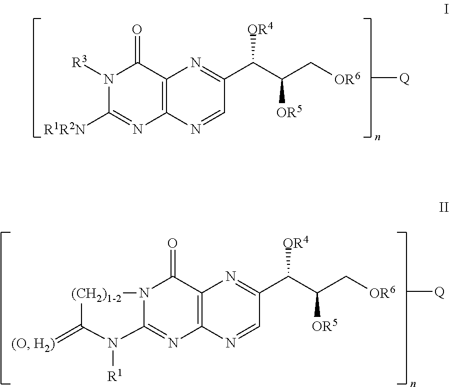

wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido

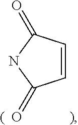

mercapto (SH) and carboxyl (CO₂H), conjugated to Q, wherein Q is a solid support, and wherein “n” is 1-20. The test sample can be plasma or serum. The immunoassay can employ a labeled anti-pterin antibody, wherein the label is an acridinium compound. The acridinium compound can be an acridinium-9-carboxamide. The acridinium-9-carboxamide can be an acridinium-9-carboxamide of formula III:

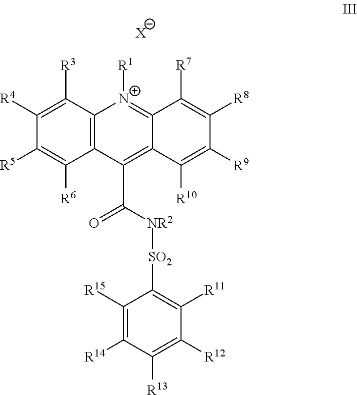

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. The acridinium compound can be an acridinium-9-carboxylate aryl ester. The acridinium-9-carboxylate aryl ester can be an acridinium-9-carboxylate aryl ester of formula IV:

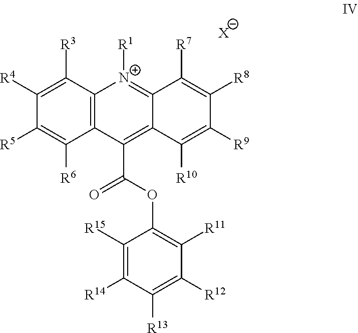

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

The test sample can be from a patient, in which case the method can further comprise diagnosing, prognosticating, or assessing the efficacy of therapeutic/prophylactic treatment of a condition comprising inflammation in the patient. If the method further comprises assessing the efficacy of therapeutic/prophylactic treatment of the patient, the method optionally further comprises modifying the therapeutic/prophylactic treatment of the patient as needed to improve efficacy.

The method can further comprise assaying, simultaneously or sequentially, in either order, by immunoassay or other assay, at least one other marker selected from the group consisting of myeloperoxidase (MPO), neutrophil gelatinase-associated lipocalin (NGAL), C-reactive protein (CRP), and calcitonin. The method can be adapted for use in an automated system or a semi-automated system.

A method of determining the presence, amount or concentration of neopterin in a test sample is provided. The method comprises assaying the test sample for neopterin by a chemiluminescent microparticle immunoassay employing an anti-neopterin antibody as a capture agent. The test sample can be plasma or serum. The immunoassay can employ labeled neopterin or a labeled anti-neopterin antibody, wherein the label is an acridinium compound. The acridinium compound can be an acridinium-9-carboxamide, such as an acridinium-9-carboxamide of formula III:

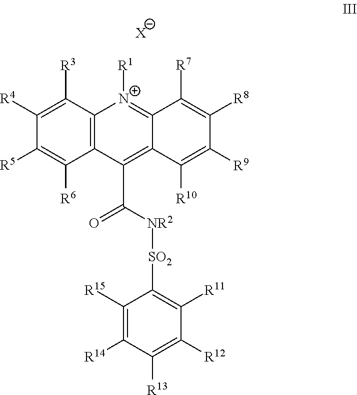

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. The acridinium compound can be an acridinium-9-carboxylate aryl ester, such as an acridinium-9-carboxylate aryl ester of formula IV:

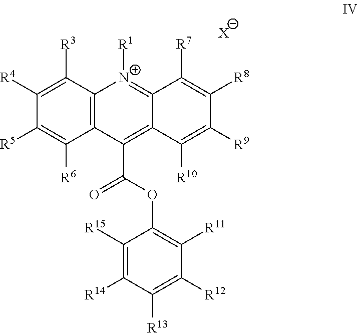

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

The test sample can be from a patient, in which case the method further comprises diagnosing, prognosticating, or assessing the efficacy of therapeutic/prophylactic treatment of a condition comprising inflammation in the patient. If the method further comprises assessing the efficacy of therapeutic/prophylactic treatment of the patient, the method optionally further comprises modifying the therapeutic/prophylactic treatment of the patient as needed to improve efficacy.

The method can further comprise assaying, simultaneously or sequentially, in either order, by chemiluminescent microparticle immunoassay or other assay, at least one other marker selected from the group consisting of MPO, NGAL, CRP, and calcitonin. The method can be adapted for use in an automated system or a semi-automated system.

Another method of determining the presence, amount or concentration of neopterin in a test sample is provided. The method comprises assaying the test sample for neopterin by an immunoassay employing as a conjugate an anti-neopterin antibody labeled with an acridinium compound. The immunoassay can be a chemiluminescent microparticle immunoassay. The test sample can be plasma or serum. The acridinium compound can be an acridinium-9-carboxamide, such as an acridinium-9-carboxamide of formula III:

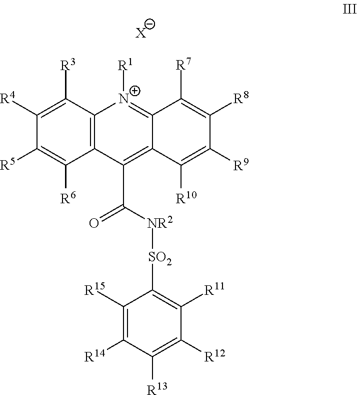

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. The acridinium compound can be an acridinium-9-carboxylate aryl ester, such as an acridinium-9-carboxylate aryl ester of formula IV:

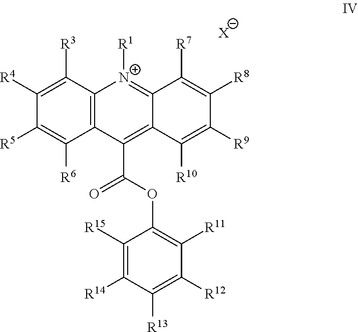

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

The test sample is from a patient and the method further comprises diagnosing, prognosticating, or assessing the efficacy of therapeutic/prophylactic treatment of a condition comprising inflammation in the patient. If the method further comprises assessing the efficacy of therapeutic/prophylactic treatment of the patient, the method optionally further comprises modifying the therapeutic/prophylactic treatment of the patient as needed to improve efficacy.

The method can further comprise assaying, simultaneously or sequentially, in either order, by immunoassay or other assay, at least one other marker selected from the group consisting of MPO, NGAL, CRP, and calcitonin. The method can be adapted for use in an automated system or a semi-automated system.

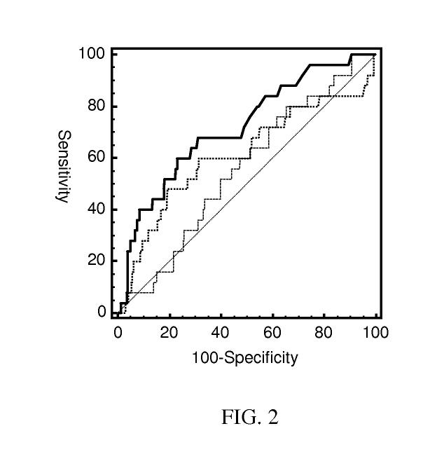

Yet another method of determining the presence, amount or concentration of neopterin in a test sample is provided. The method comprises assaying the test sample for neopterin by an immunoassay employing as a tracer neopterin labeled with an acridinium compound. The immunoassay can be a chemiluminescent microparticle immunoassay. The test sample can be plasma or serum. The acridinium compound can be an acridinium-9-carboxamide, such as an acridinium-9-carboxamide of formula III:

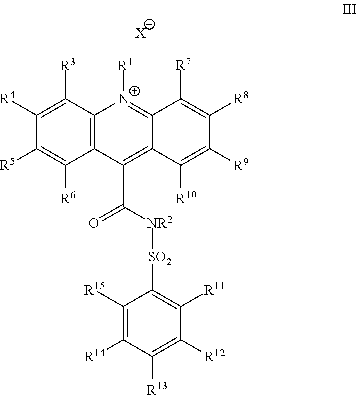

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. Examples of neopterin labeled with an acridinium compound include compound 10a, 10b, or 10c in FIG. 2, compound 25 in FIG. 7, and compound 30a, 30b, 30c, 31a, 31b, 31c, 32a, 32b, or 32c in FIG. 9. The acridinium compound can be an acridinium-9-carboxylate aryl ester, such as an acridinium-9-carboxylate aryl ester of formula IV:

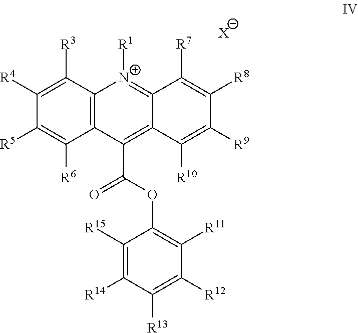

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

The test sample can be from a patient, in which case the method can further comprise diagnosing, prognosticating, or assessing the efficacy of therapeutic/prophylactic treatment of a condition comprising inflammation in the patient. If the method further comprises assessing the efficacy of therapeutic/prophylactic treatment of the patient, the method optionally further comprises modifying the therapeutic/prophylactic treatment of the patient as needed to improve efficacy.

The method can further comprise assaying, simultaneously or sequentially, in either order, by immunoassay or other assay, at least one other marker selected from the group consisting of MPO, NGAL, CRP, and calcitonin. The method can be adapted for use in an automated system or a semi-automated system.

In the context of the above methods immunoassays and other assays can be conducted using any suitable method as is known in the art. Examples include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay a capture agent that specifically binds an analyte of interest is attached to the surface of a mass spectrometry probe, such as a pre-activated protein chip array. The analyte is then specifically captured on the biochip, and the captured analyte is detected by mass spectrometry. Alternatively, the analyte can be eluted from the capture agent and detected by traditional MALDI (matrix-assisted laser desorption/ionization) or by SELDI. A chemiluminescent microparticle immunoassay, in particular one employing the ARCHITECT® automated analyzer (Abbott Laboratories, Abbott Park, Ill.), is an example of a preferred immunoassay and is exemplified herein in Example 14.

Methods well-known in the art for collecting, handling and processing urine, blood, serum and plasma, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples), pretreatment optionally can be done for mere convenience (e.g., as part of a regimen on a commercial platform).

The pretreatment reagent can be any reagent appropriate for use with the immunoassay and kits of the invention. The pretreatment optionally comprises: (a) one or more solvents (e.g., methanol and ethylene glycol) and salt, (b) one or more solvents, salt and detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., Abbott TDx Monoclonal Antibody Assay Evaluated for Measuring Cyclosporine in Whole Blood, Clin. Chem. 36: 1969-1973 (1990), and Wallemacq et al., Evaluation of the New AxSYM Cyclosporine Assay: Comparison with TDx Monoclonal Whole Blood and EMIT Cyclosporine Assays, Clin. Chem. 45: 432-435 (1999)), and/or as commercially available. Additionally, pretreatment can be done as described in Abbott's U.S. Pat. No. 5,135,875, European Pat. Pub. No. 0 471 293, U.S Pat. App. Pub. No. 2009/0325198, and U.S. Pat. App. Pub. No. 2008/0020401 (incorporated by reference in its entirety for its teachings regarding pretreatment). The pretreatment reagent can be a heterogeneous agent or a homogeneous agent.

With use of a heterogeneous pretreatment reagent, the pretreatment reagent precipitates analyte binding protein present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with a labeled specific binding partner for an analyte, such as a labeled anti-analyte antibody. The pretreatment reagent employed for such an assay typically is diluted in the pretreated test sample mixture, either before or during capture by the first specific binding partner. Despite such dilution, a certain amount of the pretreatment reagent (for example, 5 M methanol and/or 0.6 Methylene glycol) is still present (or remains) in the test sample mixture during capture.

In a heterogeneous format, after the test sample is obtained from a subject, a first mixture is prepared. The mixture contains the test sample being assessed for an analyte and a first specific binding partner, wherein the first specific binding partner and any analyte contained in the test sample form a first specific binding partner-analyte complex. Preferably, the first specific binding partner is an anti-analyte antibody or a fragment thereof. The order in which the test sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-analyte complex is formed, any unbound analyte is removed from the complex using any technique known in the art. For example, the unbound analyte can be removed by washing. Desirably, however, the first specific binding partner is present in excess of any analyte present in the test sample, such that all analyte that is present in the test sample is bound by the first specific binding partner.

After any unbound analyte is removed, a second specific binding partner is added to the mixture to form a first specific binding partner-analyte-second specific binding partner complex. The second specific binding partner is preferably an anti-analyte antibody that binds to an epitope on an analyte that differs from the epitope on the analyte bound by the first specific binding partner. Moreover, also preferably, the second specific binding partner is labeled with or contains a detectable label as described herein.

In the context of the above methods, where other than an acridinium label is used, any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, penicillinase (Malakaneh et al., Hybridoma 20(2): 117-121 (2001)) and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

A preferred acridinium compound is an acridinium-9-carboxamide, such as an acridinium-9-carboxamide of formula III:

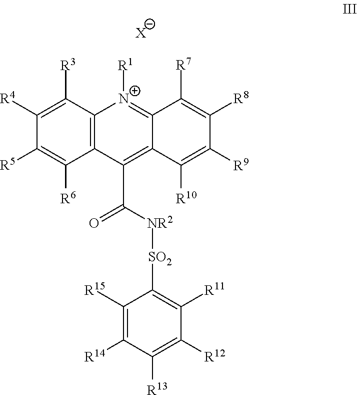

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another preferred acridinium compound is an acridinium-9-carboxylate aryl ester, such as an acridinium-9-carboxylate aryl ester of formula IV:

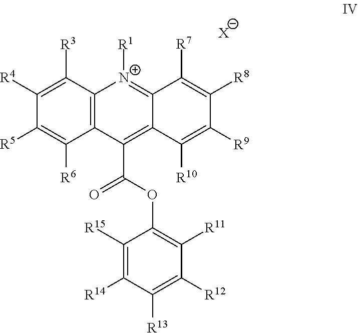

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

Acridinium-9-carboxylate aryl esters of the above formula are readily (commercially) available. A non-limiting example of a commercially available acridinium-9-carboxylate aryl ester, which is useful in the context of the disclosed methods and kits, is 10-methyl-9-(phenoxycarbonyl)-acridinium fluorosulfonate (available from Cayman Chemical, Ann Arbor, Mich.).

Chemiluminescent assays can be performed in accordance with the methods described in Adamczyk et al., Anal. Chim. Acta 579(1): 61-67 (2006). While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed. For example, the emission of light from an acridinium carboxamide can be a pseudo-flash when the reagents are added in rapid succession, such as within 5 seconds, whereas the emission of light from an acridinium carboxamide can be a long-lived glow when there is a delay, such as 20 seconds, between the addition of choline oxidase and the acridinium carboxamide.

The order in which the test sample and the specific binding partner(s) are added to form the mixture for chemiluminescent assay is not critical. If the first specific binding partner is detectably labeled with an acridinium compound, detectably labeled first specific binding partner-analyte complexes form. Alternatively, if a second specific binding partner is used and the second specific binding partner is detectably labeled with an acridinium compound, detectably labeled first specific binding partner-analyte-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound (specifically, the first specific binding partner labeled with the acridinium compound). Hydrogen peroxide can be generated in situ in a number of ways. For example, one or more hydrogen peroxide-generating enzymes can be added to the first mixture. The amount of one or more hydrogen peroxide-generating enzymes to be added to the mixture can be readily determined by one skilled in the art.

Hydrogen peroxide also can be generated electrochemically in situ (see, e.g., Agladze et al., J. Applied Electrochem. 37: 375-383 (2007); and Qiang et al., Water Research 36: 85-94 (2002)). Photochemical generation of hydrogen peroxide in situ is also possible (see, e.g., Draper et al., Archives of Environmental Contamination and Toxicology 12: 121-126 (1983)).

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of analyte in the sample can be quantified. Specifically, the amount of analyte in the sample is proportional to the intensity of the signal generated. The amount of analyte present can be quantified by comparing the amount of light generated to a standard curve for analyte or by comparison to a reference standard. The standard curve can be generated using serial dilutions or solutions of known concentrations of analyte by mass spectroscopy, gravimetric methods, and other techniques known in the art.

Immunoassays generally can be conducted using any format known in the art, such as, but not limited to, a sandwich format, as further described in U.S. Provisional Patent Application No. 60/981,473 (the '473 application), which was filed on Oct. 19, 2007, and which is hereby incorporated by reference. Specifically, in one format at least two antibodies are employed to separate and quantify an analyte in a sample. More specifically, the at least two antibodies bind to certain different epitopes on an analyte forming an immune complex, which is referred to as a “sandwich.” Generally, in the immunoassays one or more agents, such as antibodies, can be used to capture the analyte in the test sample (these agents are referred to as “capture agents” and, when the capture agent is an antibody or antibodies, the agent is/are frequently referred to as a “capture antibody” or “capture antibodies”) and one or more agents, such as antibodies, can be used to bind a detectable (namely, quantifiable) label to the sandwich (these agents are referred to as “detection agents” and, when the detection agent is/are an antibody or antibodies, the agent is/are frequently referred to as “detection antibody,” “detection antibodies,” “conjugate” or “conjugates”).

Generally speaking, a sample being tested for (for example, suspected of containing) an analyte can be contacted with at least one capture agent or capture antibody (or capture agents or capture antibodies) and at least one detection agent or detection antibody (which can be a second detection agent/antibody or a third detection agent/antibody) either simultaneously or sequentially and in any order. For example, the test sample can be first contacted with at least one capture agent/antibody and then (sequentially) with at least one detection agent/antibody. Alternatively, the test sample can be first contacted with at least one detection agent/antibody and then (sequentially) with at least one capture agent/antibody. In yet another alternative, the test sample can be contacted simultaneously with a capture agent/antibody and a detection agent/antibody.

In the sandwich assay format, a sample suspected of containing an analyte is first brought into contact with an at least one capture agent/antibody under conditions that allow the formation of a capture agent (or capture antibody)/analyte complex. If more than one capture agent/antibody is used, a multiple capture agent (or capture antibody)/analyte complex is formed. In a sandwich assay, the capture agents, preferably the at least one capture agent/antibody, are used in molar excess amounts of the maximum amount of analyte expected in the test sample. For example, from about 5 μg to about 1 mg of agent/antibody per mL of buffer (e.g., microparticle coating buffer) can be used.

Competitive inhibition immunoassays, which are often used to measure small analytes because binding by only one agent/antibody is required, comprise sequential and classic formats. In a sequential competitive inhibition immunoassay a capture agent, such as a capture monoclonal antibody, that binds to an analyte of interest is coated onto a well of a microtiter plate. When the sample containing the analyte of interest is added to the well, the analyte of interest binds to the capture agent/monoclonal antibody. After washing, a known amount of labeled (e.g., biotin or horseradish peroxidase (HRP)) analyte is added to the well. A substrate for an enzymatic label is necessary to generate a signal. An example of a suitable substrate for HRP is 3,3′,5,5′-tetramethylbenzidine (TMB). After washing, the signal generated by the labeled analyte is measured and is inversely proportional to the amount of analyte in the sample. In a classic competitive inhibition immunoassay a capture agent, such as a capture monoclonal antibody, that binds to an analyte of interest is coated onto a well of a microtiter plate. However, unlike the sequential competitive inhibition immunoassay, the sample and the labeled analyte are added to the well at the same time. Any analyte in the sample competes with labeled analyte for binding to the capture agent/monoclonal antibody. After washing, the signal generated by the labeled analyte is measured and is inversely proportional to the amount of analyte in the sample.

Optionally, prior to contacting the test sample with the at least one capture agent/antibody (for example, the first capture antibody), the at least one capture agent/antibody can be bound to a solid support, which facilitates the separation of the capture agent (or antibody)/analyte complex from the test sample. The substrate to which the capture agent/antibody is bound can be any suitable solid support or solid phase that facilitates separation of the capture agent (or antibody)-analyte complex from the sample. Examples include a well of a plate, such as a microtiter plate, a test tube, a porous gel (e.g., silica gel, agarose, dextran, or gelatin), a polymeric film (e.g., polyacrylamide), beads (e.g., polystyrene beads or magnetic beads), a strip of a filter/membrane (e.g., nitrocellulose or nylon), microparticles (e.g., latex particles, magnetizable microparticles (e.g., microparticles having ferric oxide or chromium oxide cores and homo- or hetero-polymeric coats and radii of about 1-10 microns). The substrate can comprise a suitable porous material with a suitable surface affinity to bind antigens and sufficient porosity to allow access by detection antibodies. A microporous material is generally preferred, although a gelatinous material in a hydrated state can be used. Such porous substrates are preferably in the form of sheets having a thickness of about 0.01 to about 0.5 mm, preferably about 0.1 mm. While the pore size may vary quite a bit, preferably the pore size is from about 0.025 to about 15 microns, more preferably from about 0.15 to about 15 microns. The surface of such substrates can be activated by chemical processes that cause covalent linkage of an antibody to the substrate. Irreversible binding, generally by adsorption through hydrophobic forces, of the antigen or the antibody to the substrate results; alternatively, a chemical coupling agent or other means can be used to bind covalently the agent/antibody to the substrate, provided that such binding does not interfere with the ability of the agent/antibody to bind to the analyte.

Alternatively, the capture agent/antibody can be bound with microparticles, which have been previously coated with streptavidin or biotin (e.g., using Power-BindTM-SA-MP streptavidin-coated microparticles (Seradyn, Indianapolis, Ind.)) or anti-species-specific monoclonal antibodies. If necessary, the substrate can be derivatized to allow reactivity with various functional groups on the antibody. Such derivatization requires the use of certain coupling agents, examples of which include, but are not limited to, maleic anhydride, N-hydroxysuccinimide, and 1-ethyl-3-(3-dimethylaminopropyl) carbodiimide. If desired, one or more capture agents, such as antibodies, each of which is specific for an analyte can be attached to solid phases in different physical or addressable locations (e.g., such as in a biochip configuration (see, e.g., U.S. Pat. No. 6,225,047, Int'l Pat. App. Pub. No. WO 99/51773; U.S. Pat. No. 6,329,209; Int'l Pat. App. Pub. No. WO 00/56934, and U.S. Pat. No. 5,242,828). If the capture agent is attached to a mass spectrometry probe as the solid support, the amount of analyte bound to the probe can be detected by laser desorption ionization mass spectrometry. Alternatively, a single column can be packed with different beads, which are derivatized with the one or more capture agents, thereby capturing the analyte in a single place (see, antibody-derivatized, bead-based technologies, e.g., the xMAP technology of Luminex (Austin, Tex.)).

After the test sample being assayed for analyte is brought into contact with at least one capture agent/antibody (for example, the first capture antibody), the mixture is incubated in order to allow for the formation of a first capture agent (or antibody (or multiple agents/antibodies))-analyte complex. The incubation can be carried out at a pH of from about 4.5 to about 10.0, at a temperature of from about 2° C. to about 45° C., and for a period from at least about one (1) minute to about eighteen (18) hours, preferably from about 1 to about 24 minutes, most preferably for about 4 to about 18 minutes. The immunoassay described herein can be conducted in one step (meaning the test sample, at least one capture antibody and at least one detection antibody are all added sequentially or simultaneously to a reaction vessel) or in more than one step, such as two steps, three steps, etc.

After formation of the (first or multiple) capture agent (or antibody)/analyte complex, the complex is then contacted with at least one detection agent/antibody under conditions, which allow for the formation of a (first or multiple) capture agent (or antibody)/analyte/(first or multiple) detection agent (or antibody) complex. If the capture agent (or antibody)/analyte complex is contacted with more than one detection antibody, then a (first or multiple) capture antibody/analyte/multiple detection agent/antibody complex is formed. As with the capture agent/antibody, when the at least one detection antibody is brought into contact with the capture agent (or antibody)/analyte complex, a period of incubation under conditions similar to those described above is required for the formation of the (first or multiple) capture agent (or antibody)/analyte/(first or multiple) detection agent (or antibody) complex. Preferably, at least one detection agent/antibody contains a detectable label. The detectable label can be bound to the at least one detection agent/antibody prior to, simultaneously with, or after the formation of the (first or multiple) capture agent (or antibody)/analyte/(first or multiple) detection agent (or antibody) complex. Any detectable label known in the art can be used (see discussion above, including Polak and Van Noorden (1997) and Haugland (1996)).

The detectable label can be bound to the agents/antibodies either directly or through a coupling agent. An example of a coupling agent that can be used is EDAC (1-ethyl-3-(3-dimethylaminopropyl) carbodiimide, hydrochloride), which is commercially available from Sigma-Aldrich, St. Louis, Mo. Other coupling agents that can be used are known in the art. Methods for binding a detectable label to an agent/antibody are known in the art. Additionally, many detectable labels can be purchased or synthesized that already contain end groups that facilitate the coupling of the detectable label to the antibody, such as CPSP-Acridinium Ester (i.e., 9-[N-tosyl-N-(3-carboxypropyl)]-10-(3-sulfopropyl)acridinium carboxamide) or SPSP-Acridinium Ester (i.e., N10-(3-sulfopropyl)-N-(3-sulfopropyl)-acridinium-9-carboxamide).

The (first or multiple) capture agent (or antibody)/analyte/(first or multiple) detection agent (or antibody) complex can be, but does not have to be, separated from the remainder of the test sample prior to quantification of the label. For example, if the at least one capture agent/antibody (e.g., the first capture antibody) is bound to a solid support, such as a well or a bead, separation can be accomplished by removing the fluid (of the test sample) from contact with the solid support. Alternatively, if the at least one capture agent/antibody is bound to a solid support, it can be simultaneously contacted with the test sample and the at least one detection agent/antibody to form a first (or multiple) capture agent (or antibody)/analyte/first (or multiple) detection agent (or antibody) complex, followed by removal of the fluid (test sample) from contact with the solid support. If the at least one capture agent/antibody is not bound to a solid support, then the (first or multiple) capture agent (or antibody)/analyte/(first or multiple) detection agent (or antibody) complex does not have to be removed from the test sample for quantification of the amount of the label.

After formation of the labeled capture agent (or antibody)/analyte/detection agent (or antibody) complex, the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified by detecting the light emitted either visually or by using luminometers, x-ray film, high-speed photographic film, a CCD camera, etc. Once the amount of the label in the complex has been quantified, the concentration of analyte thereof in the test sample is determined by use of a standard curve that has been generated using serial dilutions of analyte of known concentration. Other than using serial dilutions of analyte, the standard curve can be generated gravimetrically, by mass spectroscopy, and by other techniques known in the art.

In a chemiluminescent microparticle assay employing the ARCHITECT® analyzer, the conjugate diluent pH should be about 6.0+/−0.2, the microparticle coating buffer should be maintained at room temperature (i.e., at about 17 to about 27° C.), the microparticle coating buffer pH should be about 6.5+/−0.2, and the microparticle diluent pH should be about 7.8+/−0.2. Solids preferably are less than about 0.2%, such as less than about 0.15%, less than about 0.14%, less than about 0.13%, less than about 0.12%, or less than about 0.11%, such as about 0.10%.

FPIAs are based on competitive binding immunoassay principles. A fluorescently labeled compound, when excited by a linearly polarized light, will emit fluorescence having a degree of polarization inversely proportional to its rate of rotation. When a fluorescently labeled tracer-antibody complex is excited by a linearly polarized light, the emitted light remains highly polarized because the fluorophore is constrained from rotating between the time light is absorbed and the time light is emitted. When a “free” tracer compound (I.e., a compound that is not bound to an antibody) is excited by linearly polarized light, its rotation is much faster than the corresponding tracer-antibody conjugate produced in a competitive binding immunoassay. FPIAs are advantageous over RIAs inasmuch as there are no radioactive substances requiring special handling and disposal. In addition, FPIAs are homogeneous assays that can be easily and rapidly performed.

Commercially available anti-analyte antibodies can be used in the methods and kits described herein. Anti-neopterin antibodies, for example, are available from Antibodies-online GmbH, Atlanta, Ga., and AbD Serotec, Oxford, UK. Preferably, such commercially available antibodies are used as detection antibodies.

Any suitable control/calibrator composition can be used in the methods and kits described herein. The control composition generally comprises analyte and any desirable additives. In this regard, when neopterin, for example, is the analyte being assayed in accordance with the methods described herein, commercially available neopterin can be used in the control/calibrator compositions. Neopterin is available from Schircks Laboratories, Jona, Switzerland. When anti-neopterin antibody, for example, is the analyte, the above-described commercially available antibodies can be used in the control/calibrator compositions.

Generally, a predetermined level can be employed as a benchmark against which to assess results obtained upon assaying a test sample for analyte. Generally, in making such a comparison, the predetermined level is obtained by running a particular assay a sufficient number of times and under appropriate conditions such that a linkage or association of analyte presence, amount or concentration with a particular stage or endpoint of a condition comprising inflammation can be made. Typically, the predetermined level is obtained with assays of reference subjects (or populations of subjects). The analyte measured can include fragments thereof, degradation products thereof, and/or enzymatic cleavage products thereof. Serum levels of neopterin above 10 nmol/L are generally regarded as elevated (Berdowska et al. (2001), supra).

In particular, with respect to a predetermined level as employed for monitoring disease progression and/or treatment, the amount or concentration of an analyte may be “unchanged,” “favorable” (or “favorably altered”), or “unfavorable” (or “unfavorably altered”). “Elevated” or “increased” refers to an amount or a concentration in a test sample that is higher than a typical or normal level or range (e.g., predetermined level), or is higher than another reference level or range (e.g., earlier or baseline sample). The term “lowered” or “reduced” refers to an amount or a concentration in a test sample that is lower than a typical or normal level or range (e.g., predetermined level), or is lower than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal level or range for an analyte is defined in accordance with standard practice. Because the levels of analyte in some instances will be very low, a so-called altered level or alteration can be considered to have occurred when there is any net change as compared to the typical or normal level or range, or reference level or range, which cannot be explained by experimental error or sample variation. Thus, the level measured in a particular sample will be compared with the level or range of levels determined in similar samples from a so-called normal subject. In this context, a “normal subject” is an individual with no detectable condition comprising inflammation, for example, and a “normal” (sometimes termed “control”) patient or population is/are one(s) that exhibit(s) no detectable condition comprising inflammation, for example. Furthermore, given that neopterin is not routinely found at a high level in the majority of the human population, a “normal subject” can be considered an individual with no substantial detectable increased or elevated amount or concentration of neopterin, and a “normal” (sometimes termed “control”) patient or population is/are one(s) that exhibit(s) no substantial detectable increased or elevated amount or concentration of neopterin. An “apparently normal subject” is one in which neopterin has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having, or at risk of having, a condition comprising inflammation (see discussion of conditions in “Background”; all references cited therein are hereby specifically incorporated by reference herein for their teachings regarding same).

The method of assay can also involve the assay of other markers and the like as discussed above. For example, the method of assay can also involve the assay of MPO, NGAL, CRP, and/or calcitonin.

Accordingly, the methods described herein also can be used to determine whether or not a subject has or is at risk of developing a condition comprising inflammation. Specifically, such a method can comprise the steps of:

(a) determining the concentration or amount in a test sample from a subject of neopterin (e.g., using the methods described herein, or methods known in the art); and

(b) comparing the concentration or amount of neopterin determined in step (a) with a predetermined level, wherein, if the concentration or amount of neopterin determined in step (a) is favorable with respect to a predetermined level, then the subject is determined not to have or be at risk for a condition comprising inflammation. However, if the concentration or amount of neopterin determined in step (a) is unfavorable with respect to the predetermined level, then the subject is determined to have or be at risk for a condition comprising inflammation.

Additionally, provided herein is method of monitoring the progression of disease in a subject. Optimally the method comprising the steps of:

(a) determining the concentration or amount in a test sample from a subject of neopterin;

(b) determining the concentration or amount in a later test sample from the subject of neopterin; and

(c) comparing the concentration or amount of neopterin as determined in step (b) with the concentration or amount of neopterin determined in step (a), wherein if the concentration or amount determined in step (b) is unchanged or is unfavorable when compared to the concentration or amount of neopterin determined in step (a), then the disease in the subject is determined to have continued, progressed or worsened. By comparison, if the concentration or amount of neopterin as determined in step (b) is favorable when compared to the concentration or amount of neopterin as determined in step (a), then the disease in the subject is determined to have discontinued, regressed or improved.

Optionally, the method further comprises comparing the concentration or amount of neopterin as determined in step (b), for example, with a predetermined level. Further, optionally the method comprises treating the subject with one or more pharmaceutical compositions for a period of time if the comparison shows that the concentration or amount of neopterin as determined in step (b), for example, is unfavorably altered with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions. Next, the concentration or amount in a first test sample from a subject of neopterin is determined (e.g., using the methods described herein or as known in the art). After the concentration or amount of neopterin is determined, optionally the concentration or amount of neopterin is then compared with a predetermined level. If the concentration or amount of neopterin as determined in the first test sample is lower than the predetermined level, then the subject is not treated with one or more pharmaceutical compositions. However, if the concentration or amount of neopterin as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about seven (7) days to about two years, preferably from about fourteen (14) days to about one (1) year).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of neopterin is determined in the second or subsequent test sample is determined (e.g., using the methods described herein or as known in the art). The concentration or amount of neopterin as determined in each of the second and subsequent test samples is then compared with the concentration or amount of neopterin as determined in the first test sample (e.g., the test sample that was originally optionally compared to the predetermined level). If the concentration or amount of neopterin as determined in step (c) is favorable when compared to the concentration or amount of neopterin as determined in step (a), then the disease in the subject is determined to have discontinued, regressed or improved, and the subject should continue to be administered the one or pharmaceutical compositions of step (b). However, if the concentration or amount determined in step (c) is unchanged or is unfavorable when compared to the concentration or amount of neopterin as determined in step (a), then the disease in the subject is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received to decrease or lower said subject's neopterin level.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years after the first test sample from the subject is obtained. When used to monitor disease progression, the above assay can be used to monitor the progression of disease in subjects suffering from acute conditions. Acute conditions, also known as critical care conditions, refer to acute, life-threatening diseases or other critical medical conditions involving, for example, the cardiovascular system or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., about 1 minute, about 5 minutes, about 10 minutes, about 15 minutes, about 30 minutes, about 45 minutes, about 60 minutes, about 2 hours, about 3 hours, about 4 hours, 4 about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days), and the initial assay likewise is generally done within a shorter timeframe, e.g., about minutes, hours or days of the onset of the disease or condition.

The assays also can be used to monitor the progression of disease in subjects suffering from chronic or non-acute conditions. Non-critical care or, non-acute conditions, refers to conditions other than acute, life-threatening disease or other critical medical conditions involving, for example, the cardiovascular system and/or excretory system. Typically, non-acute conditions include those of longer-term or chronic duration. For non-acute conditions, repeat monitoring generally is done with a longer timeframe, e.g., hours, days, weeks, months or years (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years), and the initial assay likewise generally is done within a longer time frame, e.g., about hours, days, months or years of the onset of the disease or condition.

Furthermore, the above assays can be performed using a first test sample obtained from a subject where the first test sample is obtained from one source, such as urine, serum or plasma. Optionally the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject.

Moreover, the present disclosure also relates to methods of determining whether a subject predisposed to or suffering from a condition comprising inflammation will benefit from treatment. In particular, the disclosure relates to neopterin companion diagnostic methods and products. Thus, the method of “monitoring the treatment of disease in a subject” as described herein further optimally also can encompass selecting or identifying candidates for therapy.

Thus, in particular embodiments, the disclosure also provides a method of determining whether a subject having, or at risk for, a condition comprising inflammation is a candidate for therapy. Generally, the subject is one (i) who has experienced some sign or symptom of a condition comprising inflammation, (ii) who has actually been diagnosed as having, or being at risk for, a condition comprising inflammation, and/or (iii) who demonstrates an unfavorable concentration or amount of neopterin as described herein.

The method optionally comprises an assay as described herein, where analyte is assessed before and following treatment of a subject with one or more pharmaceutical compositions (e.g., particularly with a pharmaceutical related to a mechanism of action involving neopterin), with immunosuppressive therapy, or by immunoabsorption therapy, or where analyte is assessed following such treatment and the concentration or the amount of analyte is compared against a predetermined level. An unfavorable concentration of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

The method of assay also can be used to identify a compound that ameliorates a condition comprising inflammation. For example, a cell that expresses neopterin can be contacted with a candidate compound. The level of expression of neopterin in the cell contacted with the compound can be compared to that in a control cell using the method of assay described herein.

### Kit

A kit for assaying a test sample for a pterin is provided. The kit comprises (i) a pterin of formula I or II conjugated to Q, wherein Q is a solid support, as a capture agent and (ii) instructions for assaying the test sample for a pterin by immunoassay.

A kit for assaying a test sample for neopterin also is provided. The kit comprises (i) anti-neopterin antibody as a capture agent and (ii) instructions for assaying the sample for neopterin by chemiluminescent microparticle immunoassay.

Another kit for assaying a test sample for neopterin is provided. The kit comprises (i) an anti-neopterin antibody labeled with an acridinium compound as a conjugate and (ii) instructions for assaying the test sample for neopterin by immunoassay.

Yet another kit for assaying a test sample for neopterin is provided. The kit comprises (i) neopterin labeled with an acridinium compound as a tracer and (ii) instructions for assaying the test sample for neopterin by immunoassay.

The instructions in the above kits can be in paper form or computer-readable form, such as a disk, CD, DVD, or the like. Alternatively or additionally, the kit can comprise a calibrator or control, e.g., purified, and optionally lyophilized, analyte, and/or at least one container (e.g., tube, microtiter plates or strips, which can be already coated with a capture agent (or antibody)) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying analyte.

Any antibodies, which are provided in the kit, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc or chip.

### Adaptation of Kit and Method

The kit (or components thereof), as well as the method of assays described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (i.e., capture agent, such as a capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT®, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture agent/antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection agent/antibody labeled with alkaline phosphatase (or other label). Within the fluid pouch of the cartridge is an aqueous reagent that includes p-aminophenol phosphate.

In operation, a sample suspected of containing analyte is added to the holding chamber of the test cartridge and the cartridge is inserted into the I-STAT® reader. After the detection agent/antibody has dissolved into the sample, a pump element within the cartridge forces the sample into a conduit containing the chip. Here it is oscillated to promote formation of the sandwich between the capture agent/antibody, analyte, and the labeled detection agent/antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of analyte in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an I-STAT® cartridge format, using a nucleic acid sequence linked to the signal antibody as a signal amplifier.

### Acridinium-Labeled Anti-Neopterin Antibody

An anti-neopterin antibody labeled with an acridinium compound is provided. The antibody can be labeled with acridinium in accordance with methods described herein. The acridinium compound can be an acridinium-9-carboxamide, such as an acridinium-9-carboxamide of formula III:

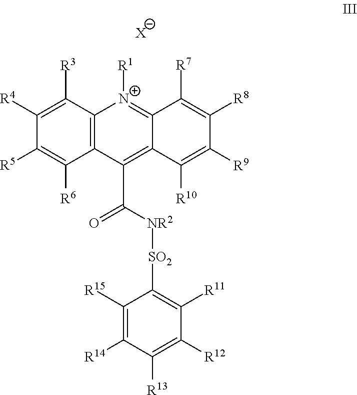

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. The acridinium compound can be an acridinium-9-carboxylate aryl ester, such as an acridinium-9-carboxylate aryl ester of formula IV:

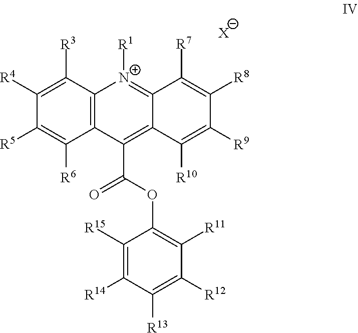

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion.

### Synthetic Production of Antibodies

Once sequenced, polypeptides, such as a monoclonal antibody, which specifically binds to an analyte (e.g, a pterin, such as neopterin), can be synthesized using methods known in the art, such as, for example, exclusive solid phase synthesis, partial solid phase synthesis, fragment condensation, and classical solution synthesis. See, e.g., Merrifield, J. Am. Chem. Soc. 85: 2149 (1963). On solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories (Richmond, Calif.), and the preparation of the hydroxymethyl resin is described by Bodonszky et al., Chem. Ind. (London) 38: 1597 (1966). The benzhydrylamine (BHA) resin has been described by Pietta and Marshall, Chem. Comm. 650 (1970) and is commercially available from Beckman Instruments, Inc. (Palo Alto, Calif.) in the hydrochloride form. Automated peptide synthesizers are commercially available, as are services that make peptides to order.

Thus, the polypeptides can be prepared by coupling an alpha-amino protected amino acid to the chloromethylated resin with the aid of, for example, cesium bicarbonate catalyst, according to the method described by Gisin, Helv. Chim. Acta. 56: 1467 (1973). After the initial coupling, the alpha-amino protecting group is removed by a choice of reagents including trifluoroacetic acid (TFA) or hydrochloric acid (HCl) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, and acetyl), aromatic urethane type protecting groups (e.g., benzyloxycarbonyl (Cbz) and substituted Cbz), aliphatic urethane protecting groups (e.g., t-butyloxycarbonyl (Boc), isopropyloxycarbonyl, and cyclohexyloxycarbonyl), and alkyl type protecting groups (e.g., benzyl and triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride and dimethyl formamide (DMF) mixtures.

After the desired amino acid sequence has been completed, the desired peptide is decoupled from the resin support by treatment with a reagent, such as TFA or hydrogen fluoride (HF), which not only cleaves the peptide from the resin, but also cleaves all remaining side chain protecting groups. When the chloromethylated resin is used, HF treatment results in the formation of the free peptide acids. When the benzhydrylamine resin is used, HF treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well-known in the art. Such procedures are also described by Stewart and Young in Solid Phase Peptide Syntheses (2nd Ed., Pierce Chemical Company, 1984).

### Recombinant Production of Antibodies

A polypeptide, such as a monoclonal antibody (or a fragment thereof), which specifically binds to an analyte (e.g., a pterin, such as a neopterin), can be recombinantly produced using methods known in the art. For example, an isolated nucleic acid comprising a nucleotide sequence encoding the antibody (or a fragment thereof) can be expressed in a host cell, and the antibody can be isolated. The isolated nucleic acid can comprise a nucleotide sequence encoding the amino acid sequence of the VH domain region, and/or a nucleotide sequence encoding the amino acid sequence of the VL domain region. The isolated nucleic acid can be synthesized with an oligonucleotide synthesizer, for example. One of ordinary skill in the art will readily appreciate that, due to the degeneracy of the genetic code, more than one nucleotide sequence can encode a given amino acid sequence. In this regard, substantially identical nucleotide sequences can be used, provided that the variant antibody as expressed competes with the non-variant antibody for the same epitope on the analyte. Codons, which are favored by a given host cell, preferably are selected for recombinant production. A nucleotide sequence encoding the VH region and/or a nucleotide sequence encoding the VL region can be combined with other nucleotide sequences using polymerase chain reaction (PCR), ligation, or ligation chain reaction (LCR) to encode an anti-analyte antibody or antigenically reactive fragment thereof. The individual oligonucleotides typically contain 5′ or 3′ overhangs for complementary assembly. Once assembled, the nucleotide sequence encoding an anti-analyte antibody or antigenically reactive fragment thereof can be inserted into a vector, operably linked to control sequences as necessary for expression in a given host cell, and introduced (such as by transformation or transfection) into a host cell. The nucleotide sequence can be further manipulated (for example, linked to one or more nucleotide sequences encoding additional immunoglobulin domains, such as additional constant regions) and/or expressed in a host cell.

Although not all vectors and expression control sequences may function equally well to express a polynucleotide sequence of interest and not all hosts function equally well with the same expression system, it is believed that those skilled in the art will be able to make a selection among these vectors, expression control sequences, optimized codons, and hosts without any undue experimentation. For example, in selecting a vector, the host must be considered because the vector must be able to replicate in it or be able to integrate into the chromosome. The vector's copy number, the ability to control that copy number, and the expression of any other proteins encoded by the vector, such as antibiotic markers, should also be considered. In selecting an expression control sequence, a variety of factors also can be considered. These include, but are not limited to, the relative strength of the sequence, its controllability, and its compatibility with the nucleotide sequence encoding the anti-analyte antibody, particularly with regard to potential secondary structures. Hosts should be selected by consideration of their compatibility with the chosen vector, their codon usage, their secretion characteristics, their ability to fold the polypeptide correctly, their fermentation or culture requirements, their ability (or lack thereof) to glycosylate the protein, and the ease of purification of the products encoded by the nucleotide sequence, etc.

The recombinant vector can be an autonomously replicating vector, namely, a vector existing as an extrachromosomal entity, the replication of which is independent of chromosomal replication (such as a plasmid). Alternatively, the vector can be one which, when introduced into a host cell, is integrated into the host cell genome and replicated together with the chromosome(s) into which it has been integrated.

The vector is preferably an expression vector in which the polynucleotide sequence encoding the anti-analyte antibody is operably linked to additional segments required for transcription of the polynucleotide sequence. The vector is typically derived from plasmid or viral DNA. A number of suitable expression vectors for expression in the host cells mentioned herein are commercially available or described in the literature. Useful expression vectors for eukaryotic hosts, include, but are not limited to, vectors comprising expression control sequences from SV40, bovine papilloma virus, adenovirus and cytomegalovirus. Specific vectors include pcDNA3.1 (+)\Hyg (Invitrogen Corp., Carlsbad, Calif.) and pCI-neo (Stratagene, La Jolla, Calif.). Examples of expression vectors for use in yeast cells include, but are not limited to, the 2μ plasmid and derivatives thereof, the POT1 vector (see, e.g., U.S. Pat. No. 4,931,373), the pJSO37 vector (described in Okkels, Ann. New York Acad. Sci. 782: 202-207 (1996)) and pPICZ A, B or C (Invitrogen). Examples of expression vectors for use in insect cells include, but are not limited to, pVL941, pBG311 (Cate et al., Cell 45: 685-698 (1986)), and pBluebac 4.5 and pMelbac (both of which are available from Invitrogen).

Other vectors that can be used allow the nucleotide sequence encoding the anti-analyte antibody to be amplified in copy number. Such amplifiable vectors are well-known in the art. These vectors include, but are not limited to, those vectors that can be amplified by dihydrofolate reductase (DHFR) amplification (see, for example, Kaufman, U.S. Pat. No. 4,470,461; and Kaufman et al., Mol. Cell. Biol. 2: 1304-1319 (1982)) and glutamine synthetase (GS) amplification (see, for example, U.S. Pat. No. 5,122,464 and European Pat. App. Pub. No. 0 338 841).

The recombinant vector can further comprise a nucleotide sequence enabling the vector to replicate in the host cell in question. An example of such a sequence for use in a mammalian host cell is the SV40 origin of replication. Suitable sequences enabling the vector to replicate in a yeast cell are the yeast plasmid 2μ replication genes REP 1-3 and origin of replication.

The vector can also comprise a selectable marker, namely, a gene or polynucleotide, the product of which complements a defect in the host cell, such as the gene coding for DHFR or the Schizosaccharomyces pombe TPI gene (see, e.g., Russell, Gene 40: 125-130 (1985)), or one which confers resistance to a drug, such as ampicillin, kanamycin, tetracycline, chloramphenicol, neomycin, hygromycin or methotrexate. For filamentous fungi, selectable markers include, but are not limited to, amdS, pyrG, arcB, niaD and sC.

Also present in the vector are “control sequences,” which are any components that are necessary or advantageous for the expression of the anti-analyte antibody. Each control sequence can be native or foreign to the nucleotide sequence encoding the anti-analyte antibody. Such control sequences include, but are not limited to, a leader, a polyadenylation sequence, a propeptide sequence, a promoter, an enhancer or an upstream activating sequence, a signal peptide sequence, and a transcription terminator. At a minimum, the control sequences include at least one promoter operably linked to the polynucleotide sequence encoding the anti-analyte antibody.

By “operably linked” is meant the covalent joining of two or more nucleotide sequences, by means of enzymatic ligation or otherwise, in a configuration relative to one another such that the normal function of the sequences can be performed. For example, a nucleotide sequence encoding a presequence or secretory leader is operably linked to a nucleotide sequence for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in the same reading frame. Linking is accomplished by ligation at convenient restriction sites. If such sites do not exist, then synthetic oligonucleotide adaptors or linkers can be used, in conjunction with standard recombinant DNA methods.

A wide variety of expression control sequences can be used in the context of the present disclosure. Such useful expression control sequences include the expression control sequences associated with structural genes of the foregoing expression vectors as well as any sequence known to control the expression of genes of prokaryotic or eukaryotic cells or their viruses, and various combinations thereof. Examples of suitable control sequences for directing transcription in mammalian cells include the early and late promoters of SV40 and adenovirus, for example, the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, J. Mol. Biol. 196: 947-50 (1987)).

In order to improve expression in mammalian cells a synthetic intron can be inserted in the 5′ untranslated region of a polynucleotide sequence encoding the antibody or a fragment thereof. An example of a synthetic intron is the synthetic intron from the plasmid pCI-Neo (available from Promega Corporation, Madison, Wis.).

Examples of suitable control sequences for directing transcription in insect cells include, but are not limited to, the polyhedrin promoter, the P10 promoter, the baculovirus immediate early gene 1 promoter, the baculovirus 39K delayed-early gene promoter, and the SV40 polyadenylation sequence.

Examples of suitable control sequences for use in yeast host cells include the promoters of the yeast a-mating system, the yeast triose phosphate isomerase (TPI) promoter, promoters from yeast glycolytic genes or alcohol dehydrogenase genes, the ADH2-4c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulas glucoamylase, A. nidulans acetamidase, Rhizomucor miehei aspartic proteinase or lipase, the TPI1 terminator, and the ADH3 terminator.

The polynucleotide sequence encoding the antibody of interest may or may not also include a polynucleotide sequence that encodes a signal peptide. The signal peptide is present when the anti-analyte antibody is to be secreted from the cells in which it is expressed. Such signal peptide, if present, should be one recognized by the cell chosen for expression of the polypeptide. The signal peptide can be homologous or heterologous to the anti-analyte monoclonal antibody or can be homologous or heterologous to the host cell, i.e., a signal peptide normally expressed from the host cell or one which is not normally expressed from the host cell. Accordingly, the signal peptide can be prokaryotic, for example, derived from a bacterium, or eukaryotic, for example, derived from a mammalian, insect, filamentous fungal, or yeast cell.

The presence or absence of a signal peptide will, for example, depend on the expression host cell used for the production of the anti-analyte antibody. For use in filamentous fungi, the signal peptide can conveniently be derived from a gene encoding an Aspergillus sp. amylase or glucoamylase, a gene encoding a Rhizomucor miehei lipase or protease or a Humicola lanuginosa lipase. For use in insect cells, the signal peptide can be derived from an insect gene (see, e.g., Int'l Pat. App. Pub. No. WO 90/05783), such as the lepidopteran Manduca sexta adipokinetic hormone precursor (see, e.g., U.S. Pat. No. 5,023,328), the honeybee melittin (Invitrogen), ecdysteroid UDP glucosyltransferase (egt) (Murphy et al., Protein Expression and Purification 4: 349-357 (1993), or human pancreatic lipase (hpl) (Methods in Enzymology 284: 262-272 (1997)).

Specific examples of signal peptides for use in mammalian cells include murine Ig kappa light chain signal peptide (Coloma, J. Imm. Methods 152: 89-104 (1992)). Suitable signal peptides for use in yeast cells include the a-factor signal peptide from S. cerevisiae (see, e.g., U.S. Pat. No. 4,870,008), the signal peptide of mouse salivary amylase (see, e.g., Hagenbuchle et al., Nature 289: 643-646 (1981)), a modified carboxypeptidase signal peptide (see, e.g., Valls et al., Cell 48: 887-897 (1987)), the yeast BAR1 signal peptide (see, e.g., Int'l Pat. App. Pub. No. WO 87/02670), and the yeast aspartic protease 3 (YAP3) signal peptide (see, e.g., Egel-Mitani et al., Yeast 6: 127-137 (1990)).

Any suitable host can be used to produce the anti-analyte antibody, including bacteria, fungi (including yeasts), plant, insect, mammal or other appropriate animal cells or cell lines, as well as transgenic animals or plants. Examples of bacterial host cells include, but are not limited to, gram-positive bacteria, such as strains of Bacillus, for example, B. brevis or B. subtilis, Pseudomonas or Streptomyces, or gram-negative bacteria, such as strains of E. coli. The introduction of a vector into a bacterial host cell can, for instance, be effected by protoplast transformation (see, for example, Chang et al., Molec. Gen. Genet. 168: 111-115 (1979)), using competent cells (see, for example, Young et al., J. of Bacteriology 81: 823-829 (1961), or Dubnau et al., J. of Molec. Biol. 56: 209-221 (1971)), electroporation (see, for example, Shigekawa et al., Biotechniques 6: 742-751 (1988)), or conjugation (see, for example, Koehler et al., J. of Bacteriology 169: 5771-5278 (1987)).

Examples of suitable filamentous fungal host cells include, but are not limited to, strains of Aspergillus, for example, A. oryzae, A. niger, or A. nidulans, Fusarium or Trichoderma. Fungal cells can be transformed by a process involving protoplast formation, transformation of the protoplasts, and regeneration of the cell wall using techniques known to those ordinarily skilled in the art. Suitable procedures for transformation of Aspergillus host cells are described in European Pat. App. Pub. No. 238 023 and U.S. Pat. No. 5,679,543. Suitable methods for transforming Fusarium species are described by Malardier et al., Gene 78: 147-156 (1989), and Int'l Pat. App. Pub. No. WO 96/00787. Yeast can be transformed using the procedures described by Becker and Guarente, In Abelson, J. N. and Simon, M. I., editors, Guide to Yeast Genetics and Molecular Biology, Methods in Enzymology 194: 182-187, Academic Press, Inc., New York; Ito et al, J. of Bacteriology 153: 163 (1983); and Hinnen et al., PNAS USA 75: 1920 (1978).

Examples of suitable yeast host cells include strains of Saccharomyces, for example, S. cerevisiae, Schizosaccharomyces, Klyveromyces, Pichia, such as P. pastoris or P. methanolica, Hansenula, such as H. polymorpha or yarrowia. Methods for transforming yeast cells with heterologous polynucleotides and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99: 193-198 (1992), Manivasakam et al., Nucleic Acids Research 21: 4414-4415 (1993), and Ganeva et al., FEMS Microbiology Letters 121: 159-164 (1994).

Examples of suitable insect host cells include, but are not limited to, a Lepidoptora cell line, such as Spodoptera frugiperda (Sf9 or Sf21) or Trichoplusia ni cells (High Five) (see, e.g., U.S. Pat. No. 5,077,214). Transformation of insect cells and production of heterologous polypeptides are well-known to those skilled in the art.

Examples of suitable mammalian host cells include Chinese hamster ovary (CHO) cell lines, simian (e.g., Green Monkey) cell lines (COS), mouse cells (for example, NS/O), baby hamster kidney (BHK) cell lines, human cells (such as human embryonic kidney (HEK) cells (e.g., HEK 293 cells (A.T.C.C. Accession No. CRL-1573))), myeloma cells that do not otherwise produce immunoglobulin protein, and plant cells in tissue culture. Preferably, the mammalian host cells are CHO cell lines and HEK 293 cell lines. Another preferred host cell is the B3.2 cell line (e.g., Abbott Laboratories, Abbott Bioresearch Center), or another dihydrofolate reductase deficient (DHFR⁻) CHO cell line (e.g., available from Invitrogen).

Methods for introducing exogenous polynucleotides into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamine™ 2000. These methods are well-known in the art and are described, for example, by Ausbel et al. (eds.), Current Protocols in Molecular Biology, John Wiley & Sons, New York, USA (1996). The cultivation of mammalian cells is conducted according to established methods, e.g., as disclosed in Jenkins, Ed., Animal Cell Biotechnology, Methods and Protocols, Human Press Inc. Totowa, N.J., USA (1999), and Harrison and Rae, General Techniques of Cell Culture, Cambridge University Press (1997).

In the production methods, cells are cultivated in a nutrient medium suitable for production of the anti-analyte antibody using methods known in the art. For example, cells are cultivated by shake flask cultivation, small-scale or large-scale fermentation (including continuous, batch, fed-batch, or solid state fermentations) in laboratory or industrial fermentors performed in a suitable medium and under conditions allowing the anti-analyte monoclonal antibody to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection). If the anti-analyte antibody is secreted into the nutrient medium, it can be recovered directly from the medium. If the anti-analyte antibody is not secreted, it can be recovered from cell lysates.

The resulting anti-analyte antibody can be recovered by methods known in the art. For example, the anti-analyte antibody can be recovered from the nutrient medium by conventional procedures including, but not limited to, centrifugation, filtration, extraction, spray drying, evaporation, or precipitation.

The anti-analyte antibody can be purified by a variety of procedures known in the art including, but not limited to, chromatography (such as, but not limited to, ion exchange, affinity, hydrophobic, chromatofocusing, and size exclusion), electrophoretic procedures (such as, but not limited to, preparative isoelectric focusing), differential solubility (such as, but not limited to, ammonium sulfate precipitation), SDS-PAGE, or extraction (see, for example, Janson and Ryden, editors, Protein Purification, VCH Publishers, New York (1989)).

Antibody fragments are also contemplated. For example, the antibody fragment can include, but is not limited to, a Fab, a Fab′, a Fab′-SH fragment, a di-sulfide linked Fv, a single chain Fv (scFv) and a F(ab′)₂ fragment. Various techniques are known to those skilled in the art for the production of antibody fragments. For example, such fragments can be derived via proteolytic digestion of intact antibodies (see, for example, Morimoto et al., J. Biochem. Biophys. Methods 24: 107-117 (1992), and Brennan et al., Science 229: 81 (1985)) or produced directly by recombinant host cells. For example, Fab′-SH fragments can be directly recovered from E. coli and chemically coupled to form F(ab′)₂ fragments (see, e.g., Carter et al., Bio/Technology 10: 163-167 (1992)). In another embodiment, the F(ab′)₂ is formed using the leucine zipper GCN4 to promote assembly of the F(ab′)₂ molecule. Alternatively, Fv, Fab or F(ab′)₂ fragments can be isolated directly from recombinant host cell culture. Single chain variable region fragments (scFv) are made by linking light and/or heavy chain variable regions by using a short linking peptide or sequence (see, e.g., Bird et al., Science 242: 423-426 (1998)). The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art. Moreover, other forms of single-chain antibodies, such as diabodies are also contemplated by the present disclosure. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see, for example, Holliger et al., PNAS USA 90: 6444-6448 (1993); and Poljak et al., Structure 2: 1121-1123 (1994)).

The antibody and antigenically reactive fragment thereof have a variety of uses. In one aspect, the antibody (or a fragment thereof) can be used as one or more immunodiagnostic reagents. For example, the antibodies of the present disclosure can be used as one or more immunodiagnostic reagents in one or more methods for detecting the presence of analyte in a test sample. More specifically, the antibody (or antigenically reactive fragment thereof) can be used as a capture antibody or a detection antibody in an immunoassay to detect the presence of analyte, such as neopterin, in a test sample.

### Other Antibody Production Methods

Other antibodies (or fragments thereof) that specifically bind to a pterin, such as neopterin, can be made using a variety of different techniques known in the art. For example, polyclonal and monoclonal antibodies can be raised by immunizing a suitable subject (such as, but not limited to, a rabbit, a goat, a mouse, or other mammal) with an immunogenic preparation, which contains a suitable immunogen. The immunogen can be enriched/purified and isolated from a cell that produces it using affinity chromatography, immune-precipitation or other techniques, which are well-known in the art. Alternatively, immunogen can be prepared using chemical synthesis using routine techniques known in the art (such as, but not limited to, a synthesizer). The antibodies raised in the subject can then be screened to determine if the antibodies bind to the immunogen (or a fragment thereof).

The unit dose of immunogen (namely, the purified protein, tumor cell expressing the protein, or recombinantly expressed immunogen (or a fragment or a variant (or a fragment thereof) thereof) and the immunization regimen will depend upon the subject to be immunized, its immune status, and the body weight of the subject. To enhance an immune response in the subject, an immunogen can be administered with an adjuvant, such as Freund's complete or incomplete adjuvant.

Immunization of a subject with an immunogen as described above induces a polyclonal antibody response. The antibody titer in the immunized subject can be monitored over time by standard techniques such as an ELISA using an immobilized antigen.

Other methods of raising antibodies include using transgenic mice, which express human immunoglobin genes (see, for example, Int'l Pat. App. Pub. Nos. WO 91/00906, WO 91/10741, and WO 92/03918). Alternatively, human monoclonal antibodies can be produced by introducing an antigen into immune-deficient mice that have been engrafted with human antibody-producing cells or tissues (for example, human bone marrow cells, peripheral blood lymphocytes (PBL), human fetal lymph node tissue, or hematopoietic stem cells). Such methods include raising antibodies in SCID-hu mice (see, for example, Int'l Pat. App. Pub. No. WO 93/05796; U.S. Pat. No. 5,411,749; or McCune et al., Science 241: 1632-1639 (1988)) or Rag-1/Rag-2 deficient mice. Human antibody-immune deficient mice are also commercially available. For example, Rag-2 deficient mice are available from Taconic Farms (Germantown, N.Y.).

Monoclonal antibodies can be generated by immunizing a subject with an immunogen. At the appropriate time after immunization, for example, when the antibody titers are at a sufficiently high level, antibody-producing cells can be harvested from an immunized animal and used to prepare monoclonal antibodies using standard techniques. For example, the antibody-producing cells can be fused by standard somatic cell fusion procedures with immortalizing cells, such as myeloma cells, to yield hybridoma cells. Such techniques are well-known in the art, and include, for example, the hybridoma technique as originally developed by Kohler and Milstein, Nature 256: 495-497 (1975)), the human B cell hybridoma technique (Kozbar et al., Immunology Today 4: 72 (1983)), and the Epstein-Barr virus (EBV)-hybridoma technique to produce human monoclonal antibodies (Cole et al., Monoclonal Antibodies and Cancer Therapy, Alan R. Liss, Inc. pp. 77-96 (1985)). The technology for producing monoclonal antibody hybridomas is well-known to those skilled in the art.

Monoclonal antibodies also can be made by harvesting antibody-producing cells, for example, splenocytes, from transgenic mice, which express human immunoglobulin genes and which have been immunized with the immunogen. The splenocytes can be immortalized through fusion with human myelomas or through transformation with EBV. These hybridomas can be made using human B cell- or EBV-hybridoma techniques described in the art (See, for example, Boyle et al., European Pat. Pub. No. 0 614 984).

Hybridoma cells producing a monoclonal antibody, which specifically binds to the immunogen, are detected by screening the hybridoma culture supernatants by, for example, screening to select antibodies that specifically bind to the immobilized immunogen (or a fragment thereof), or by testing the antibodies as described herein to determine if the antibodies have the desired characteristics, namely, the ability to bind to immunogen (or a fragment thereof). After hybridoma cells are identified that produce antibodies of the desired specificity, the clones may be subcloned, e.g., by limiting dilution procedures, for example the procedure described by Wands et al. (Gastroenterology 80: 225-232 (1981)), and grown by standard methods.

Hybridoma cells that produce monoclonal antibodies that test positive in the screening assays described herein can be cultured in a nutrient medium under conditions and for a time sufficient to allow the hybridoma cells to secrete the monoclonal antibodies into the culture medium, to thereby produce whole antibodies. Tissue culture techniques and culture media suitable for hybridoma cells are generally described in the art (See, for example, R. H. Kenneth, in Monoclonal Antibodies: A New Dimension In Biological Analyses, Plenum Publishing Corp., New York, N.Y. (1980)). Conditioned hybridoma culture supernatant containing the antibody can then be collected. The monoclonal antibodies secreted by the subclones optionally can be isolated from the culture medium by conventional immunoglobulin purification procedures such as, for example, protein A chromatography, hydroxylapatite chromatography, gel electrophoresis, dialysis, or affinity chromatography.

Monoclonal antibodies can be engineered by constructing a recombinant combinatorial immunoglobulin library and screening the library with the immunogen or a fragment thereof. Kits for generating and screening phage display libraries are commercially available (See, for example, the Pharmacia Recombinant Phage Antibody System, Catalog No. 27-9400-01; and the Stratagene SurfZAP Phage Display Kit, Catalog No. 240612). Likewise, yeast display vectors are known in the art and are commercially available (for example, pYD1 available from Invitrogen). Briefly, the antibody library is screened to identify and isolate phages or yeast cells that express an antibody that specifically binds to the immunogen or a fragment thereof. Preferably, the primary screening of the library involves screening with an immobilized immunogen or a fragment thereof.

Following screening, the display phage or yeast is isolated and the polynucleotide encoding the selected antibody can be recovered from the display phage or yeast (for example, from the phage or yeast genome) and subcloned into other expression vectors (e.g., into Saccharomyces cerevesiae cells, for example EBY100 cells (Invitrogen)) by well-known recombinant DNA techniques. The polynucleotide can be further manipulated (for example, linked to nucleic acid encoding additional immunoglobulin domains, such as additional constant regions) and/or expressed in a host cell.

Once a monoclonal antibody that specifically binds to analyte is obtained in accordance with methods described above, it can be sequenced in accordance with methods known in the art. The antibody then can be made using recombinant DNA technology, chemical synthesis, or a combination of chemical synthesis and recombinant DNA technology as described above.

Furthermore, in some aspects of the disclosure, it may be possible to employ commercially available anti-neopterin antibodies or methods for production of anti-neopterin antibodies as described in the literature. These include, but are not limited to, those available from Antibodies-online GmbH, Atlanta, Ga., and AbD Serotec, Oxford, UK.

### Anti-Neopterin Antibody Conjugates/Complexes

A conjugate/complex comprising anti-neopterin antibody and a carrier scaffold, wherein the carrier scaffold is selected from the group consisting of a protein (e.g., bovine serum albumin (BSA)), a polysaccharide, a polynucleotide, dextran, streptavidin, and a dendrimer, wherein the ratio of antibody:carrier scaffold is greater than about 4, and wherein the anti-neopterin antibody is optionally labeled, is provided.

An above-described conjugate comprising an anti-neopterin antibody and a carrier scaffold can be prepared in accordance with methods known in the art. A conjugate is formed by covalent bonding between two species. See, e.g., Guesdon et al., J. Immunol. Methods 58 (1-2): 133-142 (1983), for an antibody conjugated to BSA; Singh, Bioconj. Chem. 9: 54-63 (1998), for an Fab′ conjugated to a dendrimer carrier scaffold; and Shih et al., Int. J. Cancer 41: 832-839 (1988), for an antibody conjugated to a dextran carrier scaffold.

An above-described complex comprising an anti-neopterin antibody and a carrier scaffold can be prepared in accordance with methods known in the art. A complex is formed by non-covalent bonding between two species. Examples of complexes include, but are not limited to, antibody and analyte, biotin and avidin, lectin and carbohydrate, complementary oligonucleotides, and the like. See, e.g., Strasburger et al., “Two-site and competitive chemiluminescent immunoassays,” In: Avidin-Biotin Technology, Wilchek and Bayer, eds., Academic Press, NY (1990), pp. 481-496, for labeling antibodies with biotin and forming complexes comprising antibody and streptavidin (the carrier scaffold); Kuijpers et al., Bioconj. Chem. 4: 94-102 (1993), for labeling antibodies with oligonucleotides and forming complexes comprising antibody and DNA carrier scaffold; and Guesdon et al., J. Immunol. Methods 39: 1-13 (1980), for conjugating antibodies with lectin and forming complexes comprising antibodies and carbohydrate carrier scaffolds.

### Pterin Conjugated to Solid Support, Protein or Detectable Label

A pterin of formula I or II:

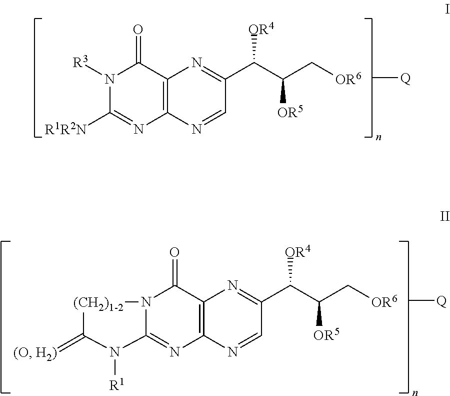

wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido

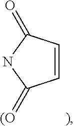

mercapto (SH) and carboxyl (CO₂H), conjugated to Q, wherein Q is a solid support, a protein, or a detectable label, and wherein “n” is 1-20, is provided. Preferably, the pterin is neopterin. The detectable label can be an acridinium compound, such as an acridinium-9-carboxamide, such as an acridinium-9-carboxamide of formula III:

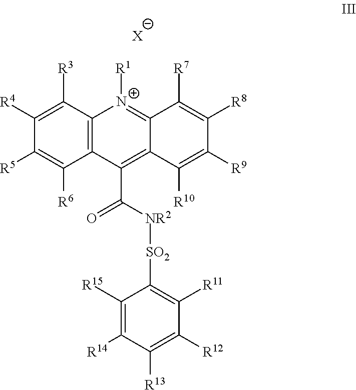

wherein R¹ and R² are each independently selected from the group consisting of alkyl, alkenyl, alkynyl, aralkyl, aryl, sulfoalkyl, carboxyalkyl and oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. Examples of pterins, i.e., neopterins, labeled with acridinium-9-carboxamide include compounds 10a, 10b, and 10c in Scheme 2, compound 25 in Scheme 7, and compounds 30a, 30b, 30c, 31a, 31b, 31c, 32a, 32b, and 32c in Scheme 9. Alternatively, the acridinium compound used to label the pterin, e.g., neopterin, can be an acridinium-9-carboxylate aryl ester, such as an acridinium-9-carboxylate aryl ester of formula IV:

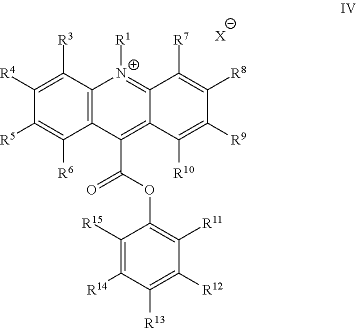

wherein R¹ is an alkyl, alkenyl, alkynyl, aryl, aralkyl, sulfoalkyl, carboxyalkyl, or oxoalkyl, and wherein R³ through R¹⁵ are each independently selected from the group consisting of hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl, and, if present, X^{⊖} is an anion. Pterins, such as neopterin, can be synthesized and labeled with acridinium compounds in accordance with the methods described herein and exemplified in the Examples.

### Conjugates of Acridinium-Labeled Pterins

A conjugate comprising (i) an above-described acridinium-labeled pterin and (ii) a carrier scaffold is also provided. The carrier scaffold can be selected from the group consisting of a protein, a polysaccharide, a polynucleotide, dextran, streptavidin, and a dendrimer, wherein the ratio of pterin:label is greater than about 10. Such conjugates can be prepared in accordance with methods described herein and exemplified in the Examples (see, e.g., Examples 6A, 6B, 11, and 13).

### Neopterin Immunogens and Conjugates Thereof

An immunogen comprising neopterin and a carrier protein, wherein the neopterin is directly conjugated to the carrier protein, is also provided. Further provided is an immunogen comprising a carrier protein and 2-N-(5-carboxypentyl)-D-neopterin, 2-N-(3-aminopropyl)-D-neopterin, 2-N-(2-carboxyethyl)-D-neopterin, 3-N-(2-carboxyethyl)-D-neopterin, or 2-N-(2-carboxyethyl)-2,3-N,N′-(1-oxopropylidinyl)-D-neopterin. The carrier protein can be bovine serum albumin (BSA), keyhole limpet hemocyanin (KLH), or thryroglobulin (TG). Such immunogens can be prepared in accordance with methods described herein and exemplified in the Examples (see, e.g., Examples 7 and 8).

A conjugate comprising an above-described immunogen and an acridinium compound is also provided. The acridinium compound can be an acridinium-9-carboxamide. Such conjugates can be prepared in accordance with methods described herein and exemplified in the Examples (see, e.g., Example 9).

### Pharmaceutical Composition

A pharmaceutical composition comprising an above-described immunogen or conjugate/complex and a pharmaceutically acceptable carrier, diluent and/or excipient can be prepared. Suitable carriers, diluents, and/or excipients are well-known in the art (see, e.g., Remington's Pharmaceutical Sciences, 20^{th} edition, Gennaro, editor, Lippincott, Williams & Wilkins, Philadelphia, Pa., 2000). Optionally, the composition further comprises another active agent and/or an adjuvant. The pharmaceutical composition is optionally part of a kit comprising one or more containers in which the antibody, another active agent and/or the adjuvant can be present in the same or different containers.

Recombinant forms of antibodies, such as chimeric and humanized antibodies, can be used in pharmaceutical compositions to minimize the response by a human patient to the antibody. When antibodies produced in non-human subjects or derived from expression of non-human antibody genes are used therapeutically in humans, they are recognized to varying degrees as foreign, and an immune response may be generated in the patient. One approach to minimize or eliminate this immune reaction is to produce chimeric antibody derivatives, namely, antibody molecules that combine a non-human animal variable region and a human constant region. Such antibodies retain the epitope binding specificity of the original monoclonal antibody but may be less immunogenic when administered to humans and, therefore, more likely to be tolerated by the patient.

Chimeric monoclonal antibodies can be produced by recombinant DNA techniques known in the art. For example, a gene encoding the constant region of a non-human antibody molecule is substituted with a gene encoding a human constant region (see, for example, Int'l Pat. App. Pub. No. PCT/US86/02269, European Pat. App. 184,187, or European Pat. App. 171,496).

A chimeric antibody can be further “humanized” by replacing portions of the variable region not involved in antigen binding with equivalent portions from human variable regions. General reviews of “humanized” chimeric antibodies can be found in Morrison, Science 229: 1202-1207 (1985), and Oi et al., BioTechniques 4: 214 (1986). Such methods include isolating, manipulating, and expressing the nucleic acid sequences that encode all or part of an immunoglobulin variable region from at least one of a heavy or light chain. The cDNA encoding the humanized chimeric antibody, or a fragment thereof, can then be cloned into an appropriate expression vector. Suitable “humanized” antibodies can be alternatively produced by complementarity determining region (CDR) substitution (see, for example, U.S. Pat. No. 5,225,539; Jones et al., Nature 321: 552-525 (1986); Verhoeyan et al., Science 239: 1534 (1988); and Beidler et al., J. Immunol. 141: 4053-4060 (1988)).

Epitope imprinting also can be used to produce a “human” antibody polypeptide dimer that retains the binding specificity of the antibodies (e.g., hamster antibodies) specific for the analyte or antigenically reactive fragment thereof. Briefly, a gene encoding a non-human variable region (VH) with specific binding to an antigen and a human constant region (CH1), is expressed in E. coli and infected with a phage library of human Vλ.Cλ genes. Phage displaying antibody fragments are then screened for binding to the analyte. Selected human Vλ genes are recloned for expression of Vλ.Cλ. chains and E. coli harboring these chains are infected with a phage library of human VHCH1 genes and the library is subject to rounds of screening with antigen-coated tubes (see, e.g., Int'l Pat. App. Pub. No. WO 93/06213).

For administration to an animal, the pharmaceutical composition can be formulated for administration by a variety of routes. For example, the composition can be formulated for oral, topical, rectal or parenteral administration or for administration by inhalation or spray. The term “parenteral” as used herein includes subcutaneous, intravenous, intramuscular, intrathecal, and intrasternal injection and infusion techniques. Various diagnostic compositions and pharmaceutical compositions suitable for different routes of administration and methods of preparing pharmaceutical compositions are known in the art and are described, for example, in “Remington: The Science and Practice of Pharmacy” (formerly “Remington's Pharmaceutical Sciences”); Gennaro, A., Lippincott, Williams & Wilkins, Philadelphia, Pa. (2000). The pharmaceutical composition can be used in the treatment of various conditions in animals, including humans.

The pharmaceutical composition preferably comprises a therapeutically or prophylactically effective amount of (i) an immunogen comprising neopterin and a carrier protein or (ii) an anti-neopterin antibody, optionally as part of a conjugate or complex. The term “therapeutically or prophylactically effective amount” as used herein refers to an amount of immunogen or anti-neopterin antibody needed to treat, ameliorate, inhibit the onset, delay or slow the progression, or prevent a targeted disease, condition, or disorder or to exhibit a detectable therapeutic or preventative effect. For anti-neopterin antibody, the therapeutically or prophylactically effective amount can be estimated initially, for example, either in cell culture assays or in animal models, usually in rodents, rabbits, dogs, pigs or primates. The animal model also can be used to determine the appropriate concentration range and route of administration. Such information then can be used to determine useful doses and routes for administration in the animal to be treated, including humans.

Other active agents can be included in the pharmaceutical composition or administered simultaneously or sequentially, in either order, with the pharmaceutical composition. If the other active agent is administered simultaneously or sequentially, in either order, with the pharmaceutical composition, such as part of a separate pharmaceutical composition, desirably the other active agent is administered at such a time relative to the administration of the pharmaceutical composition comprising an immunogen or an anti-neopterin antibody to realize at least an additive, preferably synergistic, effect.

The pharmaceutical composition can be provided as a therapeutic kit or pack. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for human or animal administration. The kit can optionally further contain one or more other active agents for use in combination with the pharmaceutical composition. The kit can optionally contain instructions or directions outlining the method of use or dosing regimen for the pharmaceutical composition.

When one or more components of the kit are provided as solutions, for example an aqueous solution, or a sterile aqueous solution, the container means can itself be an inhalant, syringe, pipette, eye dropper, or other such like apparatus, from which the solution can be administered to a subject or applied to and mixed with the other components of the kit.

The components of the kit also can be provided in dried or lyophilized form, and the kit can additionally contain a suitable solvent for reconstitution of the lyophilized components. Irrespective of the number or types of containers, the kit also can comprise an instrument for assisting with the administration of the composition to a patient. Such an instrument can be an inhalant, a syringe, a pipette, a forceps, a measuring spoon, an eye dropper, or a similar, medically approved, delivery vehicle. Accordingly, the pharmaceutical composition optionally can be part of a kit comprising one or more containers in which the immunogen, anti-neopterin antibody, another active agent and/or the adjuvant can be present in the same or different containers.

### Method of Prophylactic or Therapeutic Treatment

A method of treating a patient in therapeutic or prophylactic need of (i) an immunogen comprising neopterin and a carrier protein or (ii) an anti-neopterin antibody, optionally as part of a conjugate/complex, is also provided. The method comprises administering to the patient a pharmaceutical composition comprising a therapeutically or prophylactically effective amount of (i) an immunogen comprising neopterin and a carrier protein or (ii) an anti-neopterin antibody, optionally as part of a conjugate/complex. The composition further comprises a pharmaceutically acceptable carrier, diluent, and/or excipient. Optionally, the composition further comprises another active agent and/or an adjuvant. The method can prove useful in the treatment of a condition comprising inflammation, such as those conditions discussed in the “Background” herein, among others.

### EXAMPLES

The following examples serve to illustrate the present disclosure. The examples are not intended to limit the scope of the claimed invention in any way.

### Example 1

This example describes the preparation of 4-amino-6-hydroxy-2-methylthio-pyrimidine (compound 2 in Scheme 1).

4-Amino-6-hydroxy-2-mercapto-pyrimidine monohydrate (compound 1 in Scheme 1, 100 g, 0.62 mol; Sigma-Aldrich, Milwaukee, Wis.) was dissolved in 2.5 N aqueous sodium hydroxide (1.24 mol, 496 mL) with stirring in a 40° C. water bath. Iodomethane (176 g, 1.24 mol, 77 mL) was added dropwise to the solution. Heating was discontinued after the addition of iodomethane to the solution was complete, and the reaction mixture was allowed to cool to ambient temperature. The solid was collected by filtration, washed with water (100 mL), and dried. The filtrate was neutralized with acetic acid, and the resulting solid was collected by filtration. The collected solid was washed with water (3×100 mL), and then dried in vacuo to give compound 2 (Scheme 1, 72.5 g, 0.47 mol, 76%).

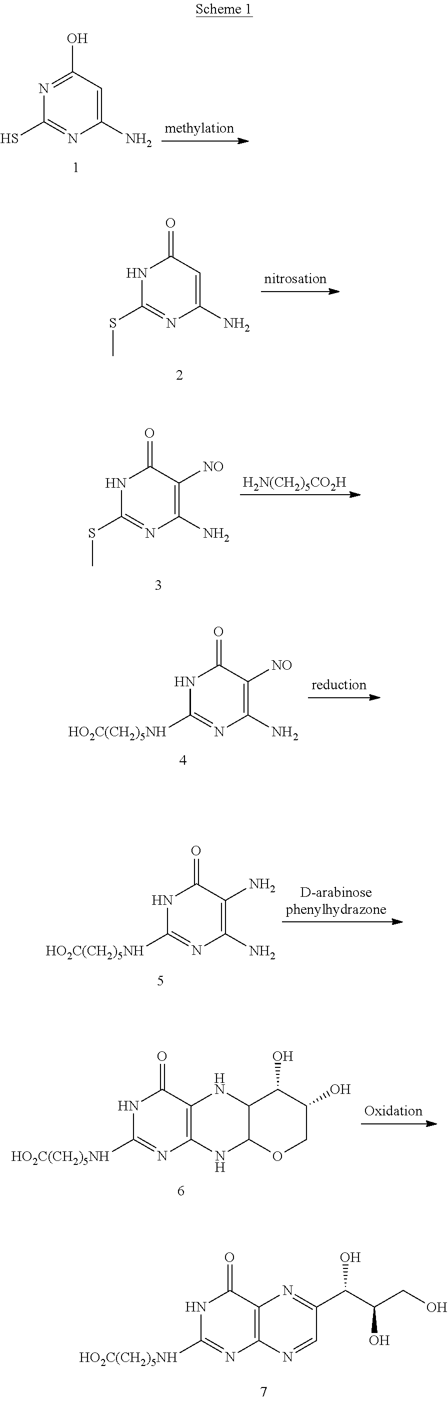

### Example 2

This example describes the preparation of 4-amino-6-hydroxy-2-methylthio-5-nitroso-pyrimidine (compound 3 in Scheme 1).

4-Amino-6-hydroxy-2-methylthio-pyrimidine (compound 2 in Scheme 1, 16.6 g, 0.0.096 mol) was suspended in water (300 mL), and 2.5 N aqueous sodium hydroxide (0.127 mol, 51 mL) was added to effect solution (compound 2, 3.2 mM). Sodium nitrite (7.8 g, 0.115 mol) was dissolved in water (50 mL), and then added to the stirred solution. Acetic acid (16 mL, 0.283 mol) was then added dropwise to give a white precipitate that turned blue with continued stirring. The solid was collected by filtration, and washed with water (100 mL), methanol (100 mL), and ether (100 mL) to give compound 3 (Scheme 1, 9 g, 0.05 mol, 52%) as a slate grey/blue solid.

### Example 3

This example describes the preparation of 6-N-(4-amino-6-hydroxy-5-nitroso-2-pyrimidyl)-aminohexanoic acid (compound 4 in Scheme 1).

6-Aminocaproic acid (18 g, 0.137 mol, 275 mol %) was added to 4-amino-6-hydroxy-2-methylthio-5-nitroso-pyrimidine (compound 3 in Scheme 1, 9 g, 0.05 mol) suspended in water (360 mL, 0.139 M). The mixture was heated to reflux to give a solution. The mixture was allowed to cool to ambient temperature, acidified to pH 2-3 with acetic acid, and chilled on ice. The solid was collected by filtration, washed with water (2×100 mL), and dried to give an orange powder (compound 4 in Scheme 1, 11.4 g, 0.042 mol, 85%).

### Example 4

This example describes the preparation of 6-N-(4,5-diamino-6-hydroxy-2-pyrimidyl)aminohexanoic acid (compound 5 in Scheme 1).

6-N-(4-Amino-6-hydroxy-5-nitroso-2-pyrimidyl)aminohexanoic acid (compound 4 in Scheme 1, 11.4 g, 0.042 mol) was suspended in aqueous sodium hydroxide (5 g, 0.125 mol, 300 mol %, 100 mL). Sodium dithionite (15.5 g, 0.088 mol, 210 mol %) was slowly added to the rapidly stirring suspension. After six hours, the reaction mixture was cooled in an ice bath and quenched with acetic acid. The solid was collected by filtration and washed with water (2×50 mL). The filtrate was retained, whereupon additional solid was collected by filtration and dried to give compound 5 (Scheme 1, 1.2 g, 0.005 mol).

### Example 5

This example describes the preparation of 2-N-(5-carboxypentyl)-D-neopterin (compound 7 in Scheme 1).

(A) D-Arabinose (0.7 g, 4.7 mmol) was dissolved in hot water (1 mL), and sodium acetate (0.76 g, 5.8 mmol) and phenylhydrazine hydrochloride (0.7 g, 4.8 mmol) were added sequentially with gentle swirling. After about 15 minutes, D-arabinose phenylhydrazone solidified. The mass was dissolved in methanol (25 mL), and added to 6-N-(4,5-diamino-6-hydroxy-2-pyrimidyl)aminohexanoic acid, (5, 1.2 g, 4.7 mmol) in water (25 mL). The pH was adjusted to 3-4 with acetic acid, and the reaction mixture was heated at reflux under nitrogen for one hour. After cooling to ambient temperature, LC/MS analysis indicated complete conversion to compound 6 (Scheme l, m/z 370 [M+H]⁺). The solution of compound 6 was treated with aqueous sodium hypochlorite (5.25% wt/vol NaOCl, 6.7 mL, 4.7 mmol). Analysis by LC/MS showed complete conversion to 2-N-(5-carboxypentyl)-D-neopterin (compound 7 in Scheme 1, m/z 368 [M+H]⁺).

(B) 6-N-(4,5-Diamino-6-hydroxy-2-pyrimidyl)aminohexanoic acid (compound 5 in Scheme 1, 0.3 g, 1.2 mmol) and D-arabinose phenylhydrazone (0.31 g, 1.3 mmol) were added to aqueous methanol (20 mL, 1:1). Triisopropylsilane (0.1 mL) and 6 N HCl (0.2 mL) were added, and the mixture was heated to 65° C. for four hours. The solution was cooled at 2° C. for 18 hours, concentrated in vacuo, and then recrystallized from methanol to give compound 6 (Scheme 1). Compound 6 was suspended in methanol (10 mL)/concentrated ammonium hydroxide (0.2 mL) and stirred in the open air for five days. LC/MS showed that the crude product oxidized to compound 7 (Scheme 1). The compound was purified by reversed-phase HPLC [YMC ODS AQ 30×150 mm; gradient elution with 0:90:10 acetonitrile/water/0.5% aq TFA to 80:10:10 over 20 minutes at 40 mL/minute]. The desired fractions were collected and lyophilized to afford 2-N-(5-carboxypentyl)-D-neopterin (compound 7, 84 mg, 23%) as a red orange solid.

### Example 6

This example describes the preparation of acridinium-9-carboxamide tracers (compounds 10a-10c in Scheme 2).

(A) A solution of 2-N-(5-carboxypentyl)-D-neopterin (compound 7 in Scheme 2, preparation described in Example 5 (A), 1 mL, 62 μmol) was evaporated to dryness in vacuo. The resulting solid was treated with N,O-bis(trimethylsilyl)acetamide/chlorotrimethylsilane in pyridine (1 mL), evaporated in vacuo, and then treated once more. The solid was dried for 12 hours under high vacuum and then taken up in aqueous N,N-dimethylformamide (1:1, 2 mL).

The acridinium-9-carboxamide (compound 9a in Scheme 2, 15 mg, 20 μmol) was added to an aliquot of the solution (1 mL). 2-N-(5-Carboxypentyl)-D-neopterin (compound 7 in Scheme 2) was activated by portion-wise addition of N-(3-dimethylaminopropyl)-N′-ethylcarbodiimide hydrochloride (EDC, 20 mg, 100 μmol) to form the active ester (compound 8 in Scheme 2), which, upon reaction with compound 9a (Scheme 2), yielded the desired acridinium-9-carboxamide tracer (compound 10a in Scheme 2; LC/MS m/z 1082 [M−H]⁻). Acridinium-9-carboxamide tracer (compound 10a in Scheme 2) was purified by HPLC [gradient elution from 10:85:5 acetonitrile/water/0.1% aq TFA to 30:75:5 acetonitrile/water/0.1% aq TFA over 10 minutes at 1 mL/min] by collecting the fractions that eluted at 9-11 minutes.

In a similar manner, the remaining aliquot of 2-N-(5-carboxypentyl)-D-neopterin (compound 7 in Scheme 2, 1 mL) was treated with acridinium-9-carboxamide (compound 9b in Scheme 2, 15 mg, 20 μmol) and EDC (20 mg, 100 μmol) to give acridinium-9-carboxamide tracer (compound 10b in Scheme 2; LC/MS m/z 1154 [M−H]⁻). Acridinium-9-carboxamide tracer (compound 10b in Scheme 2) was purified by HPLC [gradient elution from 10:85:5 acetonitrile/water/0.1% aq TFA to 30:75:5 acetonitrile/water/0.1% aq TFA over 10 minutes at 1 mL/min] by collecting the fractions that eluted at 12-14 minutes.

(B) 2-N-(5-Carboxypentyl)-D-neopterin (compound 7 in Scheme 2, preparation described in Example 5 (B), 0.020 g, 0.054 mmol), N,N,N′,N′-tetramethyl-O-(1H-benzotriazol-1-yl)uronium hexafluorophosphate (HBTU, 0.020 g, 0.054 mmol), and 1-hydroxybenzotriazole hydrate (HOBt, 0.007 g, 0.054 mmol) were combined in N,N-dimethylformamide (0.5 mL). N,N-Diisopropylethylamine (DIEA, 0.047 mL, 0.272 mmol) was added to the mixture, and the mixture was stirred for five minutes. Acridinium-9-carboxamide (compound 9c in Scheme 2, 0.035 g, 0.048 mmol) was added to the carboxyl-activated 2-N-(5-carboxypentyl)-D-neopterin (compound 8 in Scheme 2), and the reaction was stirred for 18 hours. The mixture was purified by reversed-phase HPLC [YMC ODS AQ 30×150 mm; isocratic elution with 15:75:10 acetonitrile/water/0.5% aq TFA, 40 mL/min]. The desired fractions were collected and lyophilized to afford compound 10c (Scheme 2, 16 mg, 23%) (LC/MS m/z 1172 [M+H]⁺, 586 [M+2H]⁺²).

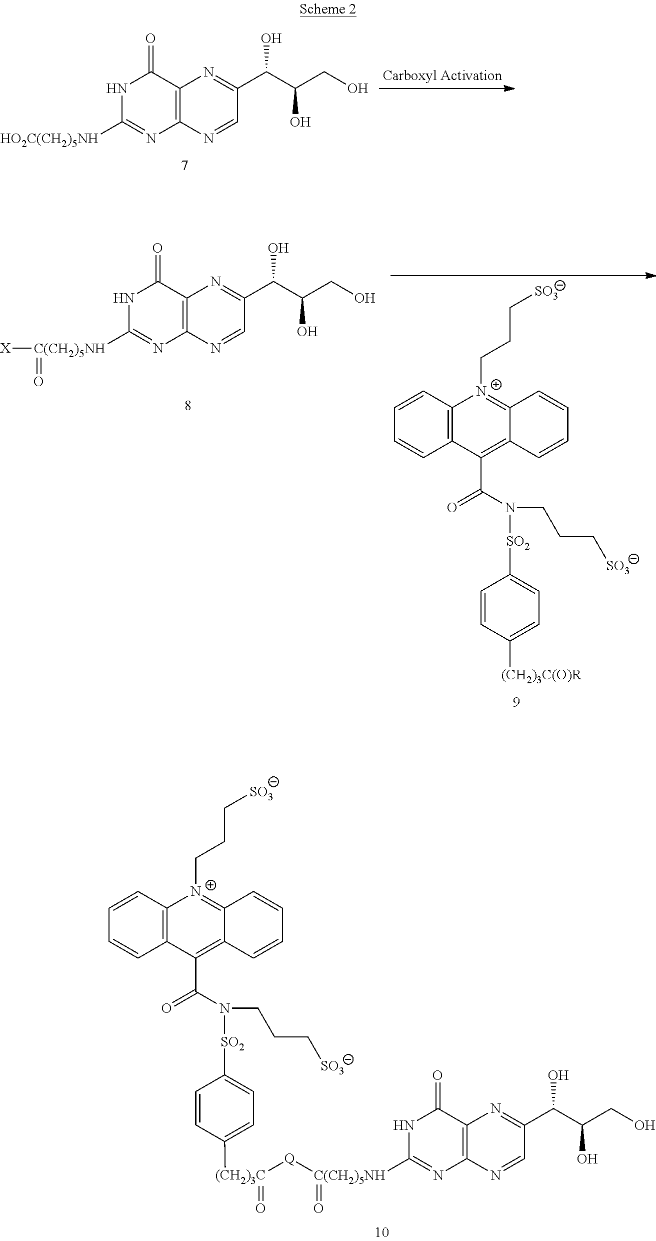

| Cmpd # | |
|---|---|
| | R |
| 9a | —NH(CH₂)₂NH₂ |
| 9b | —NH(CH₂)₆ONH₂ |
| 9c | —NH(CH₂CH₂O)₂(CH₂)₂NH₂ |
| | Q |
| 10a | —NH(CH₂)₂NH— |
| 10b | —NH(CH₂)₆ONH— |
| 10c | —NH(CH₂CH₂O)₂(CH₂)₂NH— |

### Example 7

This example describes the preparation of 2-N-(5-carboxypentyl)-D-neopterin immunogens.

2-N-(5-Carboxypentyl)-D-neopterin (compound 7 in Scheme 3, 0.017 g, 0.045 mmol) was activated by the method of (B) in Example 6 and then mixed with the carrier protein bovine serum albumin (BSA, 0.050 g) dissolved in phosphate buffer (pH 7.8, 0.1 M, 0.150 mL) for 18 hours. The immunogen was dialyzed against phosphate buffer and then water and finally lyophilized to give 26 mg of an off-white solid.

Similarly, the activated hapten is coupled to the carrier protein keyhole limpet hemocyanin (KLH) to give the corresponding 2-N-(5-carboxypentyl)-D-neopterin-KLH immunogen.

Similarly, the activated hapten is coupled to the carrier protein thyroglobulin (TG) to give the corresponding 2-N-(5-carboxypentyl)-D-neopterin-TG immunogen.

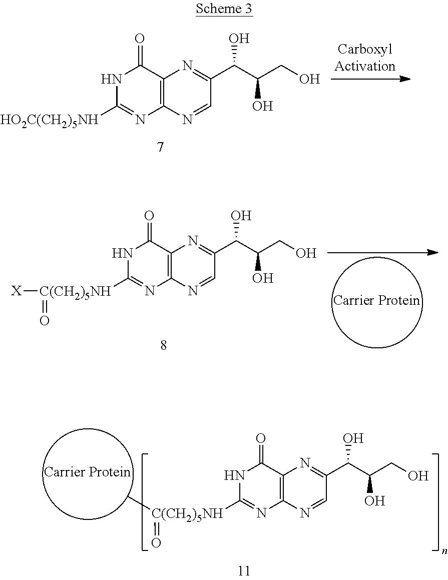

In Scheme 3, “n” can be any integer including, but not limited to, any integer from 1 to 20.

### Example 8

This example describes the preparation of D-neopterin-BSA immunogen (compound 20 in Scheme 4, wherein BSA is represented by carrier protein) by direct conjugation.

The following stock solutions were prepared: D-neopterin (53 mg) was mixed in aqueous sodium hydroxide (2 mL, 0.1 N); BSA (0.5 g) was dissolved in water (5 mL); and EDC (0.5 g) was dissolved in water (1 mL).

D-Neopterin solution (0.5 mL) and BSA solution (0.7 mL) were mixed, and aqueous sodium hydroxide (0.05 N) was added until the solution became clear. EDC solution was added in two portions (2×57 μL). After 10 minutes of mixing at ambient temperature, another 200 μL of 0.05 N aqueous sodium hydroxide were added. Mixing was continued for 3 hours at ambient temperature and then 12 hours at 2-8° C.

The D-neopterin-BSA immunogen solution was dialyzed against phosphate-buffered saline (PBS, 0.01 M, pH 7.2) using a Slide-A-Lyzer cassette (Pierce, Rockford, Ill.) with periodic injections of aqueous sodium hydroxide to maintain a clear solution. After the final buffer exchange, the D-neopterin-BSA immunogen (compound 20 in Scheme 4) solution remained clear. MALDI-TOF MS analysis indicated a mass at m/z 68769 corresponding to an average incorporation of 8.9 molecules of D-neopterin per BSA.

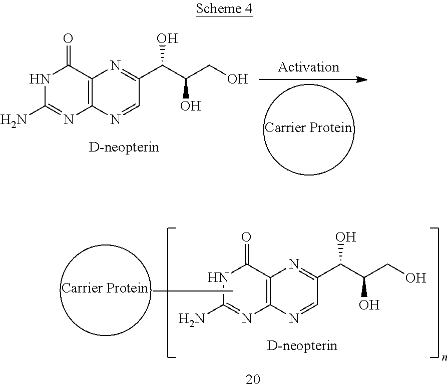

In Scheme 4, “n” can be any integer including, but not limited to, any integer from 1 to 20.

### Example 9

This example describes the preparation of D-neopterin-BSA-acridinium-9-carboxamide conjugate (compound 22 in Scheme 5).

(A) D-Neopterin-BSA immunogen (compound 20 in Scheme 5, 11.8 μL, 1 mg/mL) and acridium-9-carboxamide active ester (compound 21 in Scheme 5, 8.4 μL, 3.7 mg/mL N,N-dimethylformamide) were mixed in PBS (500 μL, 0.01 M, pH 8) for two hours, and then dialyzed against MES buffer (pH 6.5). UV analysis of the conjugate (compound 22 in Scheme 5) revealed m=3.4. Therefore, the D-neopterin-BSA-acridinium-9-carboxamide ratio was 8.9:1:3.4.

(B) D-Neopterin-BSA immunogen (compound 20 in Scheme 5, 11.8 μL, 1 mg/mL) and acridium-9-carboxamide active ester (compound 21 in Scheme 5, 2.8 μL, 3.7 mg/mL N,N-dimethylformamide) were mixed in PBS (500 μL, 0.01 M, pH 8) for two hours, and then dialyzed against MES buffer (pH 6.5). UV analysis of the conjugate (compound 22 in Scheme 5) revealed m=1.2. Therefore, the D-neopterin-BSA-acridinium-9-carboxamide ratio was 8.9:1:1.2.

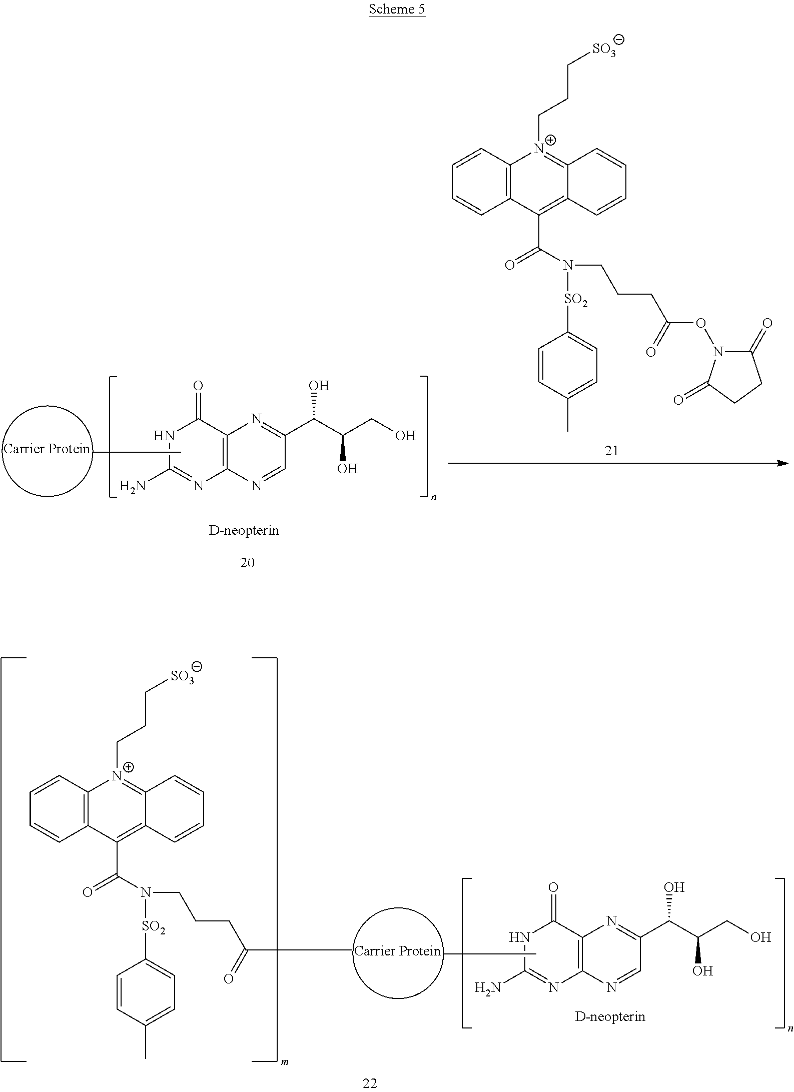

In Scheme 5, “n” and “m” independently can be any integer including, but not limited to, any integer from 1 to 20.

### Example 10

This example describes the preparation of 2-N-(3-aminopropyl)-D-neopterin hapten (compound 23 in Scheme 6).

2-N-(3-Aminopropyl)-D-neopterin 23 was prepared from compound 3 (Scheme 6) according to the procedure of Sawada et al. (Clin. Chim. Acta 138: 275-282 (1984)).

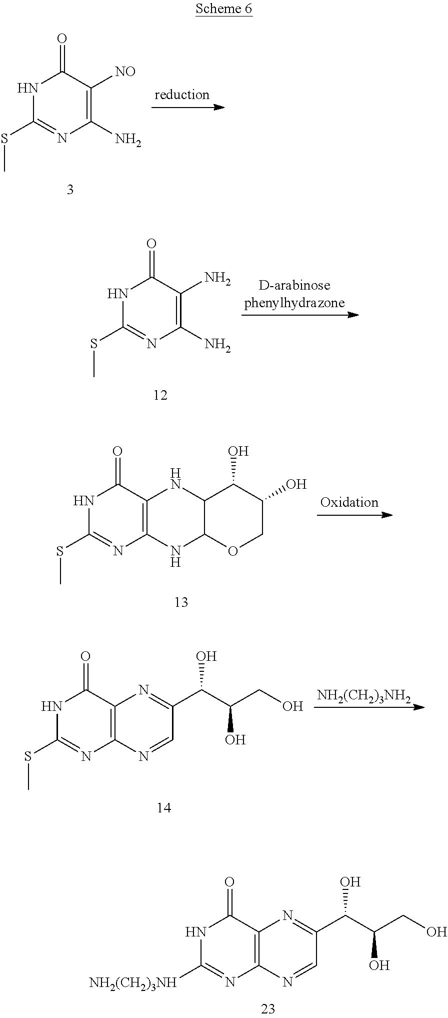

### Example 11

This example describes the preparation of 2-N-(3-aminopropyl)-D-neopterin acridinium-9-carboxamide tracers (compounds 24 and 25 in Scheme 7).

2-N-(3-Aminopropyl)-D-neopterin hapten (compound 23 in Scheme 7) is treated in aqueous N,N-dimethylformamide (pH 6-8) with the carboxyl-activated acridinium-9-carboxamide compound 9d (Scheme 7) or 9e (Scheme 7) to give 2-N-(3-aminopropyl)-D-neopterin acridinium-9-carboxamide tracer (compound 25 in Scheme 7) after purification by reversed-phase HPLC.

Similarly, 2-N-(3-aminopropyl)-D-neopterin hapten (compound 23 in Scheme 7) is treated in aqueous N,N-dimethylformamide (pH 6-8) with the carboxyl-activated acridinium-9-carboxamide compound 21 (Scheme 7) to give 2-N-(3-aminopropyl)-D-neopterin acridinium-9-carboxamide tracer (compound 24 in Scheme 7) after purification by reversed-phase HPLC.

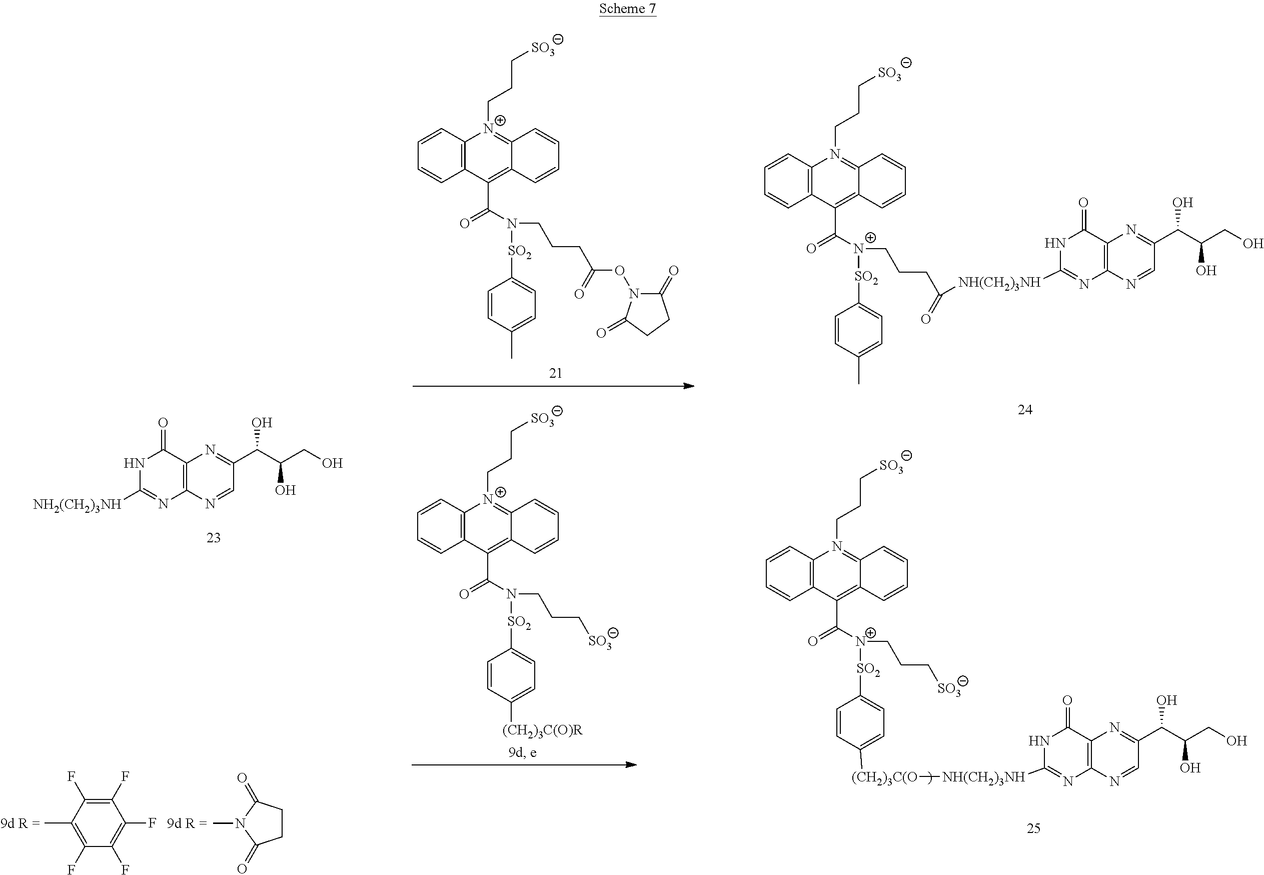

### Example 12

This example describes the preparation of 2-N-(2-carboxyethyl)-D-neopterin (compound 28 in Scheme 8), 3-N-(2-carboxyethyl)-D-neopterin (compound 27 in Scheme 8), and 2-N-(2-carboxyethyl)-2,3-N,N′-(1-oxopropylidinyl)-D-neopterin (compound 29 in Scheme 8) haptens.

D-Neopterin (3.4 g, 13.6 mmol) and acrylonitrile (6 g) are added to water (120 mL) and pyridine (20 mL) and heated at reflux. Additional portions of acrylonitrile are added over 12-24 hours until analysis indicates consumption of D-neopterin. Upon acidification with concentrated HCl, compound 26 is obtained.

Compound 26 is treated with aqueous sodium borate (0.1 M) at ambient temperature for 1-3 days, then cooled in ice, and carefully acidified to give 3-N-(2-carboxyethyl)-D-neopterin (compound 27 in Scheme 8).

Compound 26 is treated with warm aqueous sodium hydroxide for 30-90 minutes and then acidified with HCl to give 2-N-(2-carboxyethyl)-D-neopterin (compound 28 in Scheme 8).

3-N-(2-Carboxyethyl)-D-neopterin (compound 27 in Scheme 8) is treated with acrylonitrile in aqueous pyridine at reflux for two days. Additional portions of acrylonitrile are added over that time period until analysis indicates consumption of 3-N-(2-carboxyethyl)-D-neopterin (compound 27 in Scheme 8). Upon acidification with concentrated HCl, 2-N-(2-carboxyethyl)-2,3-N,N′-(1-oxopropylidinyl)-D-neopterin (compound 29 in Scheme 8) is obtained.

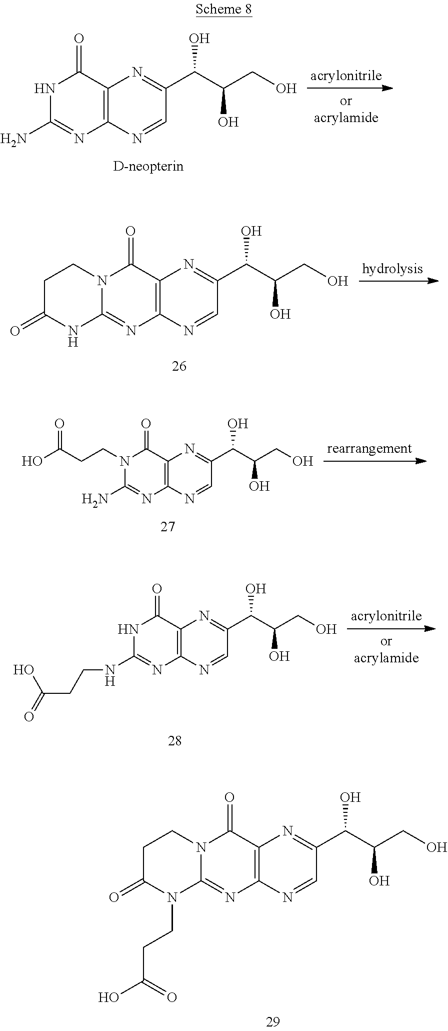

### Example 13

This example describes the preparation of 2-N-(2-carboxyethyl)-D-neopterin (compound 28 in FIG. 9), 3-N-(2-carboxyethyl)-D-neopterin (compound 27 in Scheme 9), and 2-N-(2-carboxyethyl)-2,3-N,N′-(1-oxopropylidinyl)-D-neopterin (compound 29 in Scheme 9) acridinium-9-carboxamide tracers.

D-Neopterin 2-carboxyethyl haptens (compounds 27, 28, and 29 in Scheme 9) are activated as in Example 6 and coupled with the acridinium-9-carboxamide compounds 9a-c (Scheme 2) to give D-neopterin tracers of structure 30a-c, 31a-c, and 32a-c, respectively (see Scheme 9). Each is purified by reversed-phase HPLC.

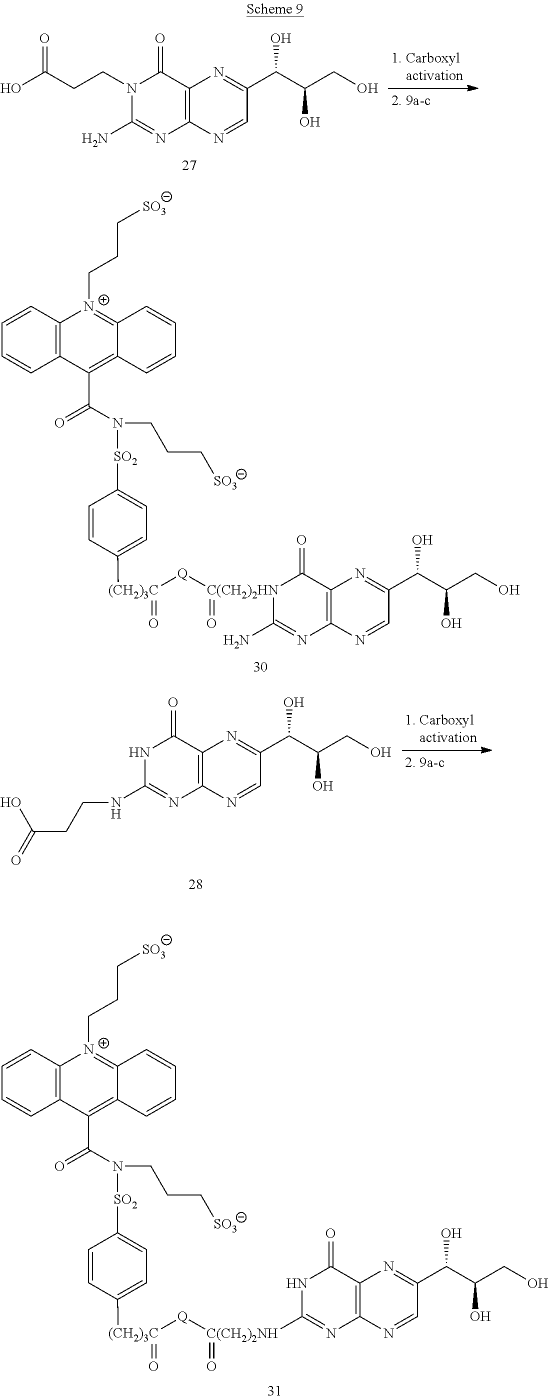

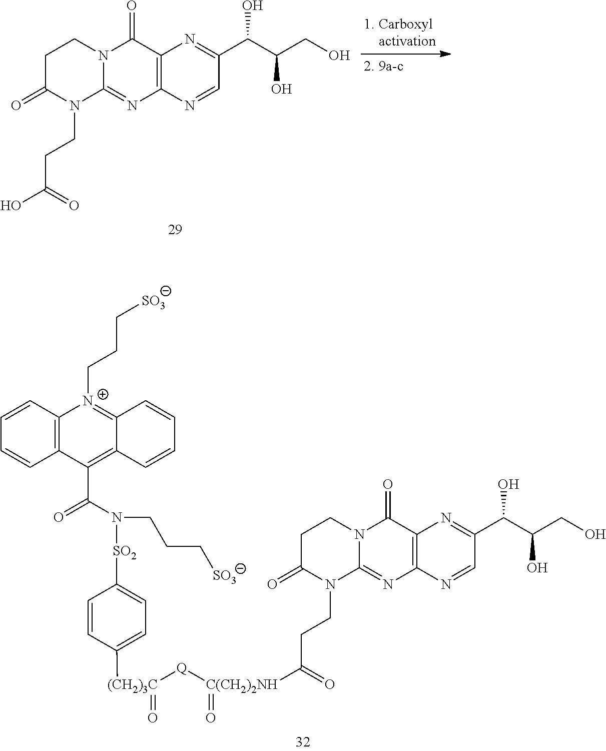

| Cmpd # | Q |
|---|---|
| 30, 31, 32 a | —NH(CH₂)₂NH— |
| 30, 31, 32 b | —NH(CH₂)₆ONH— |
| 30, 31, 32 c | —NH(CH₂)₂NH— |

### Example 14

This example describes an automated magnetic microparticle chemiluminescent immunoassay for D-neopterin.

Samples: Frozen plasma or serum from apparently healthy individuals (meaning no reported disease or symptoms of disease) were obtained from the Abbott Laboratories (Abbott Park, Ill.) specimen bank and thawed at 2-8° C. prior to use.

Microparticles: Carboxy paramagnetic microparticles (4 mL, 5% solids, nominally 5 micron diameter, Polymer Laboratories, now a part of Varian, Inc., Essex Road, Church Stretton, Shropshire, UK) were washed with 2-(N-morpholino)ethanesulfonic acid buffer (MES, 3×4 mL, pH 6.2, 50 mM) and resuspended in MES (8 mL). N-(3-Dimethylaminopropyl)-N′-ethylcarbodiimide hydrochloride (EDC, 1 mL of 6 mg/mL in water) and antibody (goat anti-mouse IgG (GAM), 1 mL, 0.8 mg/mL; or goat anti-rabbit (GAR) 1 mL, 0.8 mg/mL) were added. After mixing for 60 minutes the antibody-coated particles were magnetically sequestered, and the solution was replaced with a blocking solution consisting of 1% BSA in PBS (8 mL). After mixing for 30 minutes, the particles were washed with 1% BSA in PBS (3×, 8 mL) and resuspended in 1% BSA in PBS (8 mL). A working suspension of microparticles was prepared by dilution of the stock suspension to 0.1% solids in MES buffer (20 mM, pH 6.6) containing sucrose (13.6%) and antimicrobial agents.

Chemiluminescent D-neopterin tracer: Acridinium-9-carboxamide tracer (compounds 10a-c in Scheme 2) was diluted in a pH 4.3 diluent buffer (5 mM, 0.1% Triton X-100) to give a working solution.

Anti-D-neopterin antibody: Anti-D-neopterin IgG (mouse anti-neopterin, clone 117-14E10, Antibodies-online GmbH, Atlanta, Ga.; or rabbit anti-neopterin, AbD Serotec, Oxford, UK) were diluted in PBS (pH 7.2) to give a working solution of 0.075 μg/mL. Mouse anti-neopterin (Neopterin ELISA Kit #IB29125 IBL-America, Minneapolis, Minn.) was used as provided.

D-Neopterin Calibrators: D-Neopterin (Schircks Laboratories, Jona, Switzerland) was dissolved in human serum (Cat no. 22011-1L, Sera Con II, SeraCare Life Science, Milford, Mass.) to give calibrators at the following concentrations: 0, 2.5, 5, 10, 20, 50, 100, and 500 nM.

Assay protocol: The Assay Specific Diluent (ASD), Microparticle and Conjugate reagent positions on an ARCHITECT I2000 analyzer (Abbott Laboratories, Abbott Park, Ill.) were charged with anti-D-neopterin antibody working solution, microparticle working suspension, and chemiluminescent D-neopterin tracer solution, respectively. The samples and calibrators were placed on the Sample Carrier. After initiating the run, each sample or calibrator (150 μL) was added to an onboard reaction vessel, along with anti-D-neopterin antibody working solution (50 μL), chemiluminescent D-neopterin tracer solution, and microparticle working suspension (50 μL). After incubating for 25 minutes, the microparticles were magnetically sequestered and washed with ARCHITECT® Wash Buffer. The chemiluminescent signal from each reaction vessel was recorded after the sequential addition of ARCHITECT® Pre-Trigger solution and ARCHITECT® Trigger solution.

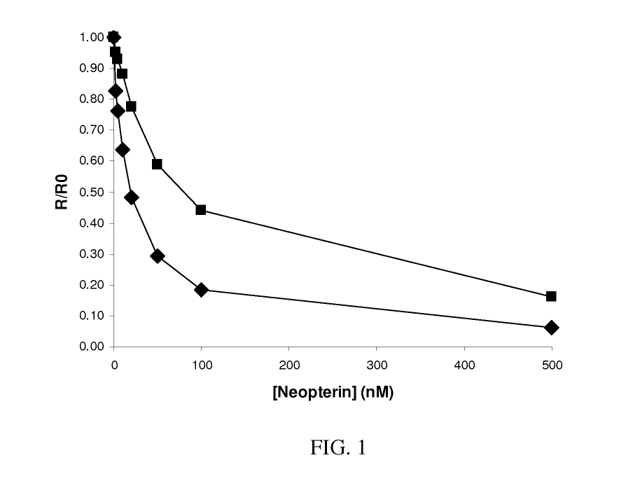

FIG. 1, which is a graph of R/R₀ vs. concentration (nM) of neopterin ([Neopterin]) comparing two different anti-neopterin antibodies, shows a comparison of the calibration curves using two different monoclonal antibodies and acridinium-9-carboxamide tracer (compound 10b in Scheme 2). Table 2 shows the results from testing the sample cohort using the IBL monoclonal antibody and acridinium-9-carboxamide tracer (compound 10b in Scheme 2).

TABLE 2

| Sample size | | |
|---|---|---|
| Lowest value | 1.8000 | |
| Highest value | 21.2000 | |
| Arithmetic mean | 8.9686 | |
| 95% CI for the mean | 7.3406 to 10.5965 | |
| Median | 8.0000 | |
| 95% CI for the median | 5.8692 to 9.7033 | |
| Variance | 22.4587 | |
| Standard deviation | 354.7391 | |
| Relative standard deviation | 0.5284 | (52.84%) |
| Standard error of the mean | 0.8010 | |
| Coefficient of Skewness | 1.0046 | (P = 0.0161) |
| Coefficient of Kurtosis | 0.6491 | (P = 0.3244) |

| | |
|---|---|
| D'Agostino-Pearson test | reject Normality |
| for Normal distribution | (P = 0.0341) |

| Percentiles | | 95% Confidence Interval |
|---|---|---|
| 2.5 | 2.1000 | |
| 25 | 5.2500 | 4.7043 to 7.2390 |
| 40 | 7.2500 | 5.2000 to 8.9709 |
| 60 | 9.2500 | 7.4764 to 10.6604 |
| 75 | 10.5750 | 9.2537 to 15.5266 |
| 90 | 17.3000 | |
| 97.5 | 20.7125 | |

Alternatively, the same reagents were used in a two-step instrument protocol in which sample/calibrator, anti-D-neopterin antibody working solution, and microparticle working suspension were incubated for 18 minutes, after which the chemiluminescent D-neopterin tracer solution was added. After incubating for 4 minutes, the microparticles were magnetically sequestered and washed with ARCHITECT® Wash Buffer. The chemiluminescent signal from each reaction vessel was recorded after the sequential addition of ARCHITECT® Pre-Trigger solution and ARCHITECT® Trigger solution.

In yet another alternative, the same reagents were used in a one-step STAT instrument protocol in which each sample or calibrator (150 μL) was added to an onboard reaction vessel along with anti-D-neopterin antibody working solution (50 μL), chemiluminescent D-neopterin tracer solution, and microparticle working suspension (50 μL). After incubating for 11 minutes, the microparticles were magnetically sequestered and washed with ARCHITECT® Wash Buffer. The chemiluminescent signal from each reaction vessel was recorded after the sequential addition of ARCHITECT® Pre-Trigger solution and ARCHITECT® Trigger solution.

In yet another alternative, the same reagents were used in a two-step STAT instrument protocol in which sample/calibrator, anti-D-neopterin antibody working solution, and microparticle working suspension were incubated for 4 minutes, after which the chemiluminescent D-neopterin tracer solution was added. After incubating for 4 minutes, the microparticles were magnetically sequestered and washed with ARCHITECT® Wash Buffer. The chemiluminescent signal from each reaction vessel was recorded after the sequential addition of ARCHITECT® Pre-Trigger solution and ARCHITECT® Trigger solution.

### Example 15

This example describes the assay of neopterin in patients with acute coronary syndrome (ACS).

Samples drawn with consent from patients (n=357) presenting at hospital emergency rooms with ischemic symptoms suggesting ACS were analyzed for neopterin, myeloperoxidase (MPO) and C-reactive protein (hsCRP). The cohort was divided on the basis of the occurrence of a major adverse cardiac event (MACE) over one year of follow-up. Samples collected from normal blood donors (ND, n=312) were also analyzed for neopterin for comparison. The results are summarized in Table 3 below.

TABLE 3

| | Neopterin | MPO Median | hsCRP Median |
|---|---|---|---|
| Cohort (n) | Median [nM] | [pM] | [mg/dL] |
| ACS_{non-MACE} (332) | 6.31 | 284 | 3.3 |
| ACS_{MACE} (25) | 11.15 | 388 | 9.7 |
| ND (312) | 6.32 | N/A | N/A |

The median concentration of neopterin at the time of admission in the ACS subgroup that experienced a MACE in the follow-up period (ACS_{MACE}) was significantly higher than either the ACS subgroup that did not experience a MACE in the follow-up period (ACS_{non-MACE}) or the normal blood donor group (ND). Upon ROC analysis (see FIG. 2, which a graph comparing the ROC for neopterin, myeloperoxidase and C-reactive protein), neopterin exhibited the highest AUC, indicating the best differentiation between the two ACS subgroups.

In the MACE subgroup, the measured neopterin concentration was greater than the cut-off derived from the ROC analysis (7.9 nM) in 68% of the cases, whereas the measured hsCRP was greater than the cut-off (13 mg/dL) in only 48% of the cases. Ninety two percent of the MACE subgroup had a neopterin level or an MPO level above their respective cut-off values, 84% had an MPO level or an hsCRP level above their respective cut-off values, and 80% had a neopterin level or an hsCRP level above their respective cut-off values, indicating the additive value of measuring neopterin in combination with MPO or hsCRP.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as defined by the appended claims.

What is claimed is:

1. A method of determining the presence, amount or concentration of a pterin in a test sample, which method comprises:
(a) assaying the test sample for a pterin by an immunoassay employing as a capture agent a pterin of formula I or II: 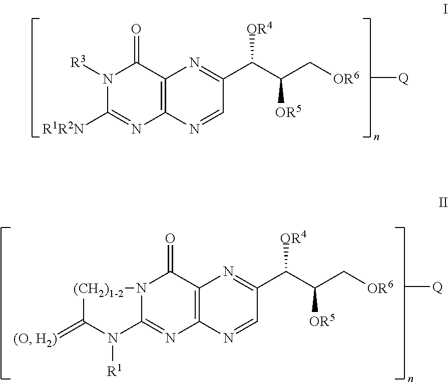
wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido 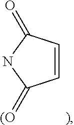
mercapto (SH) and carboxyl (CO₂H), conjugated to Q, wherein Q is a solid support, and wherein “n” is 1-20, and as a detection agent a detectably labeled anti-pterin antibody,
(b) assaying the test sample for a pterin, wherein the pterin is neopterin, by a chemiluminescent microparticle immunoassay employing an anti-neopterin antibody as a capture agent,
(c) assaying the test sample for a pterin, wherein the pterin is neopterin, by an immunoassay employing as a conjugate an anti-neopterin antibody labeled with an acridinium compound, or
(d) assaying the test sample for a pterin, wherein the pterin is neopterin, by an immunoassay employing as a tracer neopterin labeled with an acridinium compound,
whereupon the presence, amount or concentration of a pterin in the test sample is determined.

2. The method of claim 1, wherein the test sample is plasma or serum.

3. The method of claim 1, wherein the anti-pterin antibody of (a) or either of the labeled neopterin or the labeled anti-neopterin antibody of (b) is labeled with an acridinium compound.

4. The method of claim 3, wherein the acridinium compound is an acridinium-9-carboxamide.

5. The method of claim 4, wherein the acridinium-9-carboxamide is an acridinium-9-carboxamide of formula III: 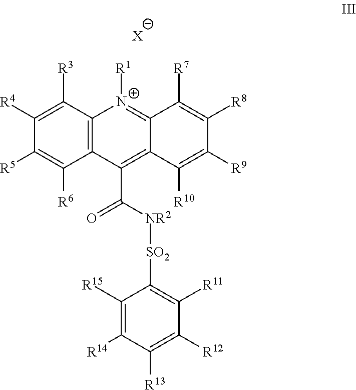
wherein R¹ and R² are each independently selected from the group consisting of: alkyl, alkenyl, alkynyl, aralkyl, aryl, sulfoalkyl, carboxyalkyl and oxoalkyl, and wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl; and
if present, X^{⊖} is an anion.

6. The method of claim 3, wherein the acridinium compound is an acridinium-9-carboxylate aryl ester.

7. The method of claim 6, wherein the acridinium-9-carboxylate aryl ester is an acridinium-9-carboxylate aryl ester of formula IV: 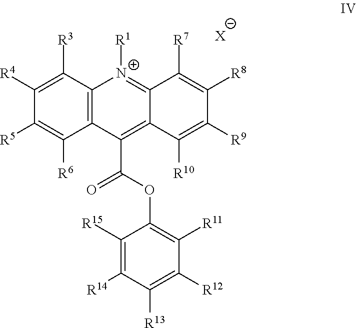
wherein R¹ is an alkyl, alkenyl, alkynyl, aryl, aralkyl, sulfoalkyl, carboxyalkyl, or oxoalkyl; and
wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl; and
if present, X^{⊖} is an anion.

8. The method of claim 1, wherein the test sample is from a patient and the method further comprises diagnosing, prognosticating, or assessing the efficacy of therapeutic/prophylactic treatment of a condition comprising inflammation in the patient, wherein, if the method further comprises assessing the efficacy of therapeutic/prophylactic treatment of the patient, the method optionally further comprises modifying the therapeutic/prophylactic treatment of the patient as needed to improve efficacy.

9. The method of claim 8, wherein the method further comprises assaying, simultaneously or sequentially, in either order, by immunoassay or other assay, at least one other marker selected from the group consisting of myeloperoxidase (MPO), neutrophil gelatinase-associated lipocalin (NGAL), C-reactive protein (CRP), and calcitonin.

10. The method of claim 1, wherein the method is adapted for use in an automated system or a semi-automated system.

11. The method of claim 1, wherein the immunoassay of (c) or (d) is a chemiluminescent microparticle immunoassay.

12. The method of claim 1, wherein the neopterin labeled with an acridinium compound is compound 10a, 10b, or 10c in Scheme 2, compound 25 in Scheme 7, or compound 30a, 30b, 30c, 31a, 31b, 31c, 32a, 32b, or 32c in Scheme 9.

13. An anti-neopterin antibody, which is labeled with an acridinium compound and which is optionally part of a conjugate/complex comprising anti-neopterin antibody and a carrier scaffold, or
an anti-neopterin antibody, which is part of a conjugate/complex comprising anti-neopterin antibody and a carrier scaffold and which is optionally detectably labeled,
wherein the carrier scaffold is selected from the group consisting of a protein, a polysaccharide, a polynucleotide, dextran, streptavidin, and a dendrimer, and wherein the ratio of antibody:carrier scaffold is greater than about 4.

14. The anti-neopterin antibody of claim 13, wherein the acridinium compound is an acridinium-9-carboxamide.

15. The anti-neopterin antibody of claim 14, wherein the acridinium-9-carboxamide is an acridinium-9-carboxamide of formula III: 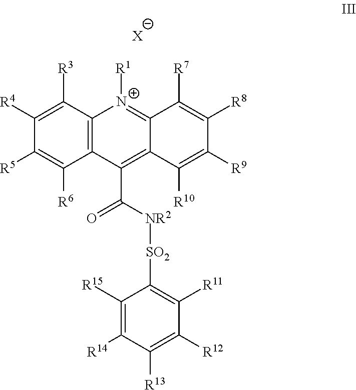
wherein R¹ and R² are each independently selected from the group consisting of: alkyl, alkenyl, alkynyl, aralkyl, aryl, sulfoalkyl, carboxyalkyl and oxoalkyl, and wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl; and
if present, X^{⊖} is an anion.

16. The anti-neopterin antibody of claim 13, wherein the acridinium compound is an acridinium-9-carboxylate aryl ester.

17. The anti-neopterin antibody of claim 16, wherein the acridinium-9-carboxylate aryl ester is an acridinium-9-carboxylate aryl ester of formula IV: 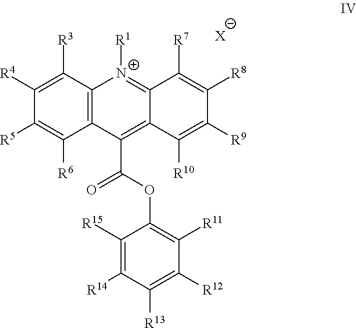
wherein R¹ is an alkyl, alkenyl, alkynyl, aryl, aralkyl, sulfoalkyl, carboxyalkyl, or oxoalkyl; and
wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl; and
if present, X^{⊖} is an anion.

18. A pterin of formula I or II: 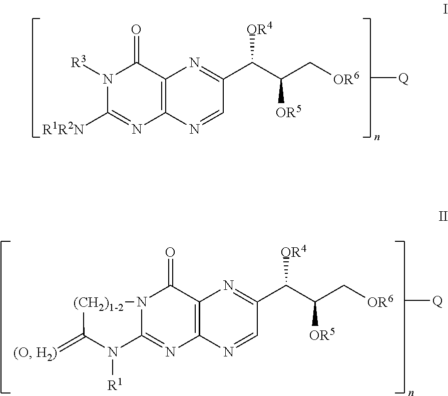
wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido 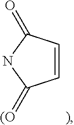
mercapto (SH) and carboxyl (CO₂H), conjugated to Q, wherein Q is a solid support, a protein, or a detectable label, and wherein “n” is 1-20.

19. The pterin of claim 18, wherein the pterin is neopterin.

20. The pterin of claim 18, wherein the detectable label is an acridinium compound, in which case the pterin is optionally part of a conjugate comprising (i) a pterin labeled with an acridinium compound and (ii) a carrier scaffold.

21. The pterin of claim 20, wherein the acridinium compound is an acridinium-9-carboxamide.

22. The pterin of claim 21, wherein the acridinium-9-carboxamide is an acridinium-9-carboxamide of formula III: 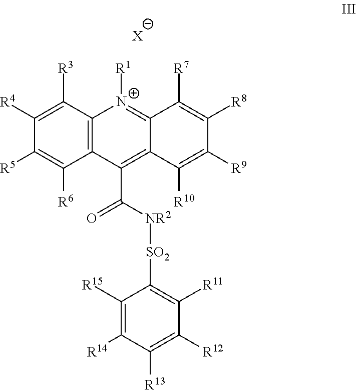
wherein R¹ and R² are each independently selected from the group consisting of: alkyl, alkenyl, alkynyl, aralkyl, aryl, sulfoalkyl, carboxyalkyl and oxoalkyl, and wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl; and
if present, X^{⊖} is an anion.

23. The pterin of claim 22, wherein the pterin labeled with an acridinium compound is compound 10a, 10b, or 10c in Scheme 2, compound 25 in Scheme 7, or compound 30a, 30b, 30c, 31a, 31b, 31c, 32a, 32b, or 32c in Scheme 9.

24. The pterin of claim 18, wherein the acridinium compound is an acridinium-9-carboxylate aryl ester.

25. The pterin of claim 24, wherein the acridinium-9-carboxylate aryl ester is an acridinium-9-carboxylate aryl ester of formula IV: 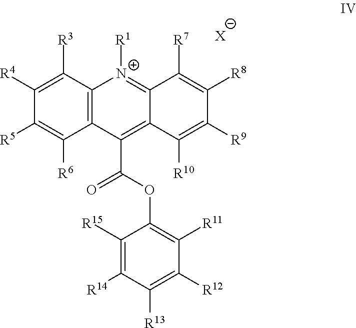
wherein R¹ is an alkyl, alkenyl, alkynyl, aryl, aralkyl, sulfoalkyl, carboxyalkyl, or oxoalkyl; and
wherein R³ through R¹⁵ are each independently selected from the group consisting of: hydrogen, alkyl, alkenyl, alkynyl, aryl, aralkyl, amino, amido, acyl, alkoxyl, hydroxyl, carboxyl, halogen, halide, nitro, cyano, sulfo, sulfoalkyl, carboxyalkyl and oxoalkyl; and
if present, X^{⊖} is an anion.

26. The conjugate of claim 20, wherein the carrier scaffold is selected from the group consisting of a protein, a polysaccharide, a polynucleotide, dextran, streptavidin, and a dendrimer, wherein the ratio of pterin:label is greater than about 10.

27. An immunogen comprising neopterin and a carrier protein, wherein the neopterin is directly conjugated to the carrier protein, wherein the immunogen is optionally part of a conjugate comprising an immunogen and an acridinium compound.

28. The immunogen of claim 27, wherein the carrier protein is bovine serum albumin (BSA), keyhole limpet hemocyanin (KLH), or thryroglobulin (TG).

29. The conjugate of claim 27, wherein the acridinium compound is an acridinium-9-carboxamide.

30. The immunogen of claim 27, wherein the neopterin is 2-N-(5-carboxypentyl)-D-neopterin, 2-N-(3-aminopropyl)-D-neopterin, 2-N-(2-carboxyethyl)-D-neopterin, 3-N-(2-carboxyethyl)-D-neopterin, or 2-N-(2-carboxyethyl)-2,3-N,N′-(1-oxopropylidinyl)-D-neopterin.

31. A kit for assaying a test sample, which kit comprises (i) a pterin of formula I or II as a capture agent: 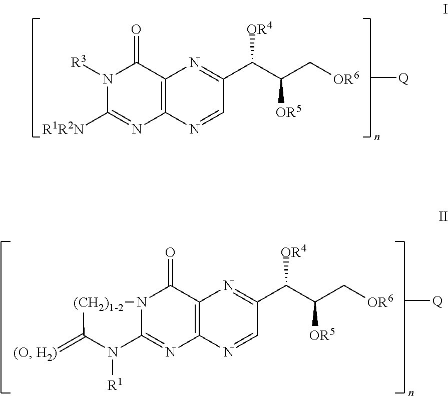
wherein R¹ through R⁶ are each independently selected from the group consisting of hydrogen or a linker of the formula —X—Y—Z, wherein X is selected from the group consisting of methylene (CH₂), carbonyl (C═O), and sulfonyl (SO₂), Y is selected from the group consisting of (CH₂)₁₋₅, (CH₂OCH₂)₁₋₅(CH₂)₁₋₂, and (CH₂)₁₋₂(C₆H₄), and Z is a reactive functional group selected from the group consisting of amino (NH₂), oxyamino (ONH₂), maleimido 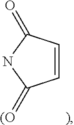
mercapto (SH) and carboxyl (CO₂H), conjugated to Q, wherein Q is a solid support, and wherein “n” is 1-20, and instructions for assaying the test sample for a pterin by immunoassay, (ii) an anti-neopterin antibody as a capture agent and instructions for assaying the test sample for neopterin by chemiluminescent microparticle immunoassay, (iii) an anti-neopterin antibody labeled with an acridinium compound as a conjugate and instructions for assaying the test sample for neopterin by immunoassay, or (iv) neopterin labeled with an acridinium compound as a tracer and instructions for assaying the test sample for neopterin by immunoassay.
